(19) United States
(12) Patent Application Publication
(10) Pub. No.: US 2015/0160224 A1
(43) Pub. Date: Jun. 11, 2015

(54) QUANTITATION OF BIOMARKERS FOR THE DETECTION OF PROSTATE CANCER

(71) Applicant: Eastern Virginia Medical School, Norfolk, VA (US)

(72) Inventor: Dean Troyer, Norfolk, VA (US)

(73) Assignee: EASTERN VIRGINIA MEDICAL SCHOOL, Norfolk, VA (US)

(21) Appl. No.: 14/397,113

(22) PCT Filed: Mar. 15, 2013
(86) PCT No.: PCT/US13/32526

§ 371 Date: Oct. 24, 2014

Related U.S. Application Data
(60) Provisional application No. 61/639,768, filed on Apr. 27, 2012.
(60) Provisional application No. 61/764,288, filed on Feb. 13, 2013.
(60) Provisional application No. 61/772,226, filed on Mar. 4, 2013.

(51) Int. Cl. G01N 33/574; G01N 33/68

(52) U.S. Cl. CPC G01N 33/57434; G01N 33/6812; G01N 33/6815; G01N 2570/00; G01N 2800/52

(57) ABSTRACT

The present invention provides biomarkers that are useful in determining whether a subject has cancer, specifically prostate cancer. Specifically, betaine, malate, proline, N-acetylaspartate, uracil, xanthine, cysteine, alanine, and N-acetylglucosamine can be used to diagnose an individual with prostate cancer, one at risk for developing prostate cancer, and/or to determine the prognosis of a subject with prostate cancer. This invention also relates to multiplexed assays for quantitating such biomarkers.

### RELATED APPLICATIONS

This application claims the benefit under 35 U.S.C. §119(e) of U.S. Provisional Application Nos. 61/639,768 filed on Apr. 27, 2012, 61/764,288 filed on Feb. 13, 2013, and 61/772,226 filed on Mar. 4, 2013, which are hereby incorporated by reference herein in their entirety.

### FIELD OF THE INVENTION

This invention relates to novel biomarkers for treating, diagnosing, and preventing prostate cancer. The invention also relates to methods of identifying, characterizing, and using such prostate cancer biomarkers. This invention also relates to multiplexed assays for quantitating biomarkers.

### BACKGROUND

Prostate cancer is one of the most common malignancies in the US (1). It is clinically heterogeneous, with a highly variable natural history (2). The discovery and widespread utilization of serum prostate specific antigen (PSA) monitoring for early detection has greatly changed the way prostate cancer is diagnosed and treated. However, PSA lacks specificity as a screening tool for prostate cancer, and there is really no lower limit of PSA that entirely excludes cancer (3). Thus, clinical decision making in prostate cancer places a significant burden upon biopsy results. Ultrasound guided needle biopsy is the standard for diagnosis however, a negative result does not exclude the presence of cancer. Both sampling and analytical variables account for false negative results. In practice, false negative results engender a need for repeat biopsies which can delay diagnosis and treatment or unnecessarily subject cancer-free men to repeat biopsies and their attendant anxiety and risk (4, 5). The heterogeneity of prostate cancer is also a significant problem and while the incidence is high, the death rates from prostate cancer are relatively low as compared to those from the other major cancers such as lung, pancreas, and colon. The Gleason grading system which has been widely adopted for prostate cancer is a predictor of outcome (6). However, a major limitation of this grading system, and a result of aggressive screening procedures, is that a majority of newly diagnosed prostate cancer cases are Gleason score 6 or 7 tumors. These moderately differentiated tumors can either be indolent or aggressive (7).

As referenced above, a determination of the prognosis of prostate cancer is guided by the Gleason grading system. In this system, a biopsy of the prostate tissue is harvested, fixed in formalin, embedded in paraffin, and then sliced and stained for viewing. The pathologist will then give the particular sample of tissue a grade or pattern based on the appearance of the tissue. The grade will range from 1 to 5, with a higher number indicating a more aggressive cancer. The pathologist gives a grade to the most common tumor pattern and then a grade to the second most common tumor pattern. These grades are added to provide the overall Gleason score. The Gleason score ranges from 2 to 10, with 10 having the worst prognosis.

The Gleason score is only one component of prostate cancer staging. The most common method of prostate cancer staging is promulgated by the American Joint Committee on Cancer and is known as the “TNM” system. There are two types of staging, the clinical stage and the pathologic stage. The clinical stage is determined prior to treatment such as surgical prostatectomy, and includes five key elements:

The extent of the primary tumor (T category)

Whether the cancer has spread to nearby lymph nodes (N category)

The absence or presence of distant metastasis (M category)

The PSA level at the time of diagnosis

The Gleason score, based on the prostate biopsy (or surgery)

T describes the size of the primary tumor. N describes whether nearby lymph nodes are involved in the cancer and M describes metastasis or spread of the cancer.

Following surgical prostatectomy, the prostate is carefully examined for assignment of pathologic stage. The “T” scale for prostate cancer is as follows:
T1: tumor present, but not detectable clinically or with imaging
   T1a: tumor was incidentally found in less than 5% of prostate tissue resected (for other reasons)
   T1b: tumor was incidentally found in greater than 5% of prostate tissue resected
   T1c: tumor was found in a needle biopsy performed due to an elevated serum PSA
T2: the tumor can be felt (palpated) on examination, but has not spread outside the prostate
   T2a: the tumor is in half or less than half of one of the prostate gland's two lobes
   T2b: the tumor is in more than half of one lobe, but not both
   T2c: the tumor is in both lobes but within the prostatic capsule
T3: the tumor has spread through the prostatic capsule (if it is only part-way through, it is still T2)
   T3a: the tumor has spread through the capsule on one or both sides
   T3b: the tumor has invaded one or both seminal vesicles
T4: the tumor has invaded other nearby structures
   This ranking, coupled with the N and M, is combined with the histological assessment from the Gleason score to determine whether definitive treatment for the cancer should be taken or watchful waiting should be chosen.

A variety of nomograms are available to assess the risk of aggressive prostate cancer including the d'Amico system (8). This assigns the following risk scores: Low-risk: PSA less than or equal to 10, Gleason score less than or equal to 6, and clinical stage T1-2a; Intermediate risk: PSA between 10 and 20, Gleason score 7, or clinical stage T2b: High-risk: PSA more than 20, Gleason score equal or larger than 8, or clinical stage T2c-3a. Definitive treatment entails radiation therapy, or prostatectomy. Therapy may also be deferred in an attempt to balance expected life span, the likelihood of treatment side effects, and quality of life. Watchful waiting (periodic clinical visits and PSA measurements) or active surveillance (periodic clinic visits and PSA measurements combined with scheduled repeat biopsies) are used when patients are comfortable with postponement of definitive therapy.

The current methods for diagnosing and making prognostic decision-making for prostate cancer have limitations in that interobserver variability occurs, especially in the setting of small tumors. This is where quantitative information would be of value to patient and physician. Therefore, new quantitative methods to assist clinicians and pathologists in both diagnostic and prognostic decision making are needed to aid in the detection and treatment of prostate cancer.

The majority of men with prostate cancer will die with their disease rather than of it (67), and there is a strong argument that screening has increased the detection of indolent tumors (68). Unfortunately, we lack clinical tools to distinguish indolent from aggressive prostate cancer (69), and it is estimated that over 1400 men need to be screened and nearly 50 men treated to prevent one prostate cancer death (70). Prostate cancers with similar microscopic features have variable clinical outcomes, reflecting genetic and biological variables of individual patients not recognized by microscopy alone (71). Unfortunately, tissue architecture is often destroyed by extraction methods required for detection of molecular tissue biomarkers. Therefore, emerging methods for biomarker discovery and validation compete with histology for the same tissue are needed. This is especially so when small needle biopsies are utilized, which is the standard of care for diagnosis of suspected prostate cancer.

Pathologic examination remains a gold standard for diagnosis, classification, and staging of tumors. This requires intact tissue while implementation of molecular tests often requires extraction methods that disrupt tissue. Metabolomics is a newer area of biospecimen analysis in which small molecules (˜2 kD) (e.g., metabolites), present in a biological sample, are extracted, detected and measured. The method has been employed in the study of the biochemical basis and mechanisms for diverse biological processes such as cancer diagnosis and monitoring progression, drug mechanism of action, drug toxicity, industrial bio-processing, etc.

In order to analyze the metabolites of a biological sample, they must be extracted completely from the sample. Existing methods of biological extraction involve destroying the sample such that it can no longer be used for other analysis (e.g., histology). Fixation of tissue samples is usually done with formalin (formaldehyde in water), followed by histologic processing and sectioning, and this is the usual work flow that produces a slide for microscopic examination by a pathologist. The drawback of this method is that formaldehyde is not an effective extractant for metabolites. Therefore, under commonly known biological extraction methods, in order to conduct histology analysis and perform metabolomics, two tissue samples are required, one for each analysis. The current state of the art is that the second biopsy would be used only for metabolomics and would not be examined histologically. Thus, it cannot be known with certainty whether the biopsy used for metabolomics contained diseased tissue.

Methods and reagents for performing metabolomics and subsequent histology on various tissue samples are described in detail in PCT Appl. No. PCT/US2011/037093 (WO2011/146683), which is herein incorporated by reference in its entirety. A preferred embodiment of that method involves contacting a single biological sample (e.g. a tissue biopsy) with a solvent (e.g. ethanol or methanol) such that the extracted biochemical can be analyzed and the extracted tissue retains its cellular architecture so that it can be subsequently analyzed using standard histological methods (including cytological analysis). Using this method, a number of tissues have been extracted, their biological chemicals analyzed, and subsequent histology preformed. See, Shuster et al. “Molecular preservation by extraction and fixation, mPREF: a method for small molecule biomarker analysis and histology on exactly the same tissue.” BMC Clinical Pathology 2011, 11:14, herein incorporated by reference in its entirety.

The term, molecular preservation by extraction and fixation (“mPREF”), refers to a process of preserving cellular structure in tissue or cell specimens whilst extracting small molecules by immersing the tissue in a solution containing an organic solvent, then subsequently processing the exact same portion of tissue using histological methods. mPREF enables both quantitation of biochemicals, including small molecule metabolites, and histological examination of the same tissue sample. mPREF permits quantitation of metabolites and histology in exactly the same tissue. This diminishes the competition of new molecular testing methods with standard histology for small amounts of tumor containing biopsy tissue. mPREF treated tissues can be used for all existing methods that use paraffin embedded tissue. The aqueous alcohol in mPREF selectively extracts small molecules from tissue, leaving macromolecules such as proteins, RNA, and DNA in place. Existing powerful in-situ methods for detecting proteins (immunohistochemistry, IHC) and RNA and DNA (fluorescence in situ hybridization, FISH) in intact tissue can continue to be used in mPREF processed tissue.

A biomarker is an organic biomolecule, the presence of which in a sample is used to determine the phenotypic status of the subject (e.g., cancer patient v. normal patient or prognosis of cancer patient). In order for the biomarker to be biologically relevant it should be differentially present in a sample taken from a subject of one phenotypic status (e.g., having a disease) as compared with another phenotypic status (e.g., not having the disease). Biomarkers, alone or in combination, provide measures of relative risk that a subject belongs to one phenotypic status or another. Therefore, they are useful as markers for disease (diagnostics), prognosis (i.e., state of the disease), therapeutic effectiveness of a drug (theranostics), drug toxicity, and predicting and identifying the immune response.

There remains a need for multiplex assays to quantitate diagnostic cancer metabolites that are isolated from biological samples in a manner that allows such samples to be further analyzed using standard histological methods. The metabolites that are identified and characterized can then be used as cancer biomarkers.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In addition, the materials, methods, and examples are illustrative only and not intended to be limiting.

### BRIEF SUMMARY OF THE INVENTION

In one embodiment, the invention provides a method for screening for prostate cancer in a subject by: (a) providing a biological sample from a subject; (b) detecting at least one biomarker in said sample, said biomarker selected from the group consisting of betaine, malate, proline, N-acetylaspartate, uracil, xanthine, cysteine, alanine, and N-acetylglucosamine; and (c) correlating said detection with a status of prostate cancer or no prostate cancer.

In a further embodiment, the invention provides a method for screening for prostate cancer in a subject by: (a) providing a biological sample from a subject; (b) detecting at least one biomarker in said sample, said biomarker selected from the group consisting of betaine, malate, proline, uracil, xanthine, cysteine, alanine, and N-acetylglucosamine; and (c) correlating said detection with a status of prostate cancer or no prostate cancer.

In a further embodiment the detecting at least one biomarker is performed by mass spectrometry.

In a still further embodiment, the biological sample is selected from the group consisting of biological fluid and tissue.

In a further embodiment, the biological fluid is whole blood, serum, plasma, or urine.

In a further embodiment, the tissue is a prostate tissue sample.

In a further embodiment, the biological sample is contacted with a solvent capable of extracting the at least one biomarker.

In a further embodiment, the solvent is methanol or ethanol.

In another embodiment, the invention provides a method of diagnosing prostate cancer in a subject by: (a) obtaining one or more test samples from a subject; (b) detecting at least one biomarker in the one or more test samples, wherein the biomarker is selected from: betaine, malate, proline, N-acetylaspartate, uracil, xanthine, cysteine, alanine, and N-acetylglucosamine; (c) quantitating the amount of the at least one biomarker; and (d) correlating the quantitation of the at least one biomarker with a diagnosis of prostate cancer.

In yet another embodiment, the invention provides a method of diagnosing prostate cancer in a subject by: (a) obtaining one or more test samples from a subject; (b) detecting at least one biomarker in the one or more test samples, wherein the biomarker is selected from: betaine, malate, proline, uracil, xanthine, cysteine, alanine, and N-acetylglucosamine; (c) quantitating the amount of the at least one biomarker; and (d) correlating the quantitation of the at least one biomarker with a diagnosis of prostate cancer, wherein the correlation takes into account the amount of the at least one biomarker in the one or more test samples compared to a control amount of the at least one biomarker.

In a further embodiment, the correlation takes into account the amount of the at least one biomarker in the one or more test samples compared to a control amount of the at least one biomarker.

In a further embodiment, the test sample is selected from the group consisting of urine, whole blood, serum, plasma, and prostate tissue.

In another embodiment, the invention provides a method of monitoring the effect of a prostate cancer drug or therapy on a subject by: (a) providing a biological sample from the subject; (b) contacting the biological sample with a solvent capable of extracting at least one prostate cancer biomarker selected from the group consisting of betaine, malate, proline, N-acetylaspartate, uracil, xanthine, cysteine, alanine, and N-acetylglucosamine; (c) quantitating the amount of the at least one prostate cancer biomarker; (d) providing the subject with an anti-prostate cancer drug or therapy; (e) quantitating the amount of the at least one prostate cancer biomarker using steps (a) and (b); and (f) correlating the two measurements with a diagnosis that the prostate cancer is regressing or progressing.

In another embodiment, the invention provides a multiplexed assay for screening for prostate cancer in a subject by: (a) providing a biological sample from a subject; (b) quantitating at least two or more biomarkers in said sample, said biomarkers selected from the group consisting of betaine, malate, proline, N-acetylaspartate, uracil, xanthine, cysteine, alanine, and N-acetylglucosamine; (c) correlating said quantitation with a status of prostate cancer or no prostate cancer.

In a further embodiment, the quantitating at least two or more biomarkers is performed by liquid chromatography in tandem with mass spectrometry.

In a further embodiment, the biological sample is selected from the group consisting of biological fluid and tissue.

In a further embodiment, the biological fluid is whole blood, serum, plasma, or urine.

In a further embodiment, the tissue is a prostate tissue sample.

In a further embodiment, the biomarkers betaine, malate, proline, N-acetylaspartate, uracil, xanthine, cysteine, alanine, and N-acetylglucosamine are quantitated in the same assay.

In a further embodiment, the biomarkers betaine, malate, proline, uracil, xanthine, cysteine, alanine, and N-acetylglucosamine are quantitated in the same assay.

In another embodiment, the invention provides a multiplexed method for detecting prostate cancer in a subject by: (a) providing a biological sample from the subject; (b) contacting the biological sample with a solvent capable of extracting two or more prostate cancer biomarkers selected from the group consisting of betaine, malate, proline, N-acetylaspartate, uracil, xanthine, cysteine, alanine, and N-acetylglucosamine; (c) quantitating the amount of the two or more biomarkers present in the biological sample; and (d) correlating the amount of the two or more biomarkers with the presence or absence of prostate cancer.

In a further embodiment, the quantitating differentiates between different stages of prostate cancer.

In a further embodiment, the quantitating is part of a diagnosis or prognosis of prostate cancer in the subject.

In a further embodiment, the solvent is methanol or ethanol.

In a further embodiment, the step of performing additional histological analysis on the extracted biological sample.

In another embodiment, the invention provides a method of diagnosing prostate cancer in a subject by: (a) obtaining one or more test samples from a subject; (b) detecting at least one biomarker in the one or more test samples, wherein the biomarker is selected from the group consisting of betaine, malate, proline, uracil, xanthine, cysteine, alanine, and N-acetylglucosamine; (c) quantitating the amount of the at least one biomarker; (d) determining the Gleason score of the one or more test samples; (e) correlating the quantitation of the at least one biomarker and the Gleason score with a relative risk of T2 versus T3 prostate cancer.

In another embodiment, the invention provides a kit for diagnosing prostate cancer in a subject with (a) a vial for collecting a biological sample from the subject; (b) a solvent for extracting biomarkers from the biological sample, the biomarkers selected from the group consisting of betaine, malate, proline, N-acetylaspartate, uracil, xanthine, cysteine, alanine, and N-acetylglucosamine; (c) instructions for performing the extraction of the biomarkers; (d) instructions for quantitating one or more of the biomarkers; (f) instructions for correlating the quantitation of the one or more biomarkers to a diagnosis of prostate cancer or normal.

### BRIEF DESCRIPTION OF THE DRAWINGS

The following figures are provided for the purpose of illustration only and are not intended to be limiting.

FIG. 1: A quantitation curve of uracil.

FIG. 2: A quantitation curve of N-acetylaspartate.

FIG. 3: A quantitation curve of xanthine.

FIG. 4: A quantitation curve of alanine.

FIG. 5: A quantitation curve of proline.

FIG. 6: A quantitation curve of betaine.

FIG. 7: A quantitation curve of cysteine.

FIG. 8: A quantitation curve of malate.

FIG. 9: Quantitation results of targeted biomarker compounds are provided in FIG. 9.

FIG. 10: FIG. 10 shows the concentration ranges where the measured values of the biomarkers of the present invention fell on the concentration standard curves. These are shaded gray.

FIG. 11: FIG. 11 shows the actual values from the 29 prostate samples (15 tumor and 14 non-tumor) that were analyzed by the current method.

FIG. 12: FIG. 12 shows the concentration ranges where the measured values of the biomarkers of the present invention fell on the concentration standard curves. These are shaded gray.

FIG. 13: A quantitation curve of uracil.

FIG. 14: A quantitation curve of N-acetylaspartate.

FIG. 15: A quantitation curve of xanthine.

FIG. 16: A quantitation curve of alanine.

FIG. 17: A quantitation curve of proline.

FIG. 18: A quantitation curve of betaine.

FIG. 19: A quantitation curve of cysteine.

FIG. 20: A quantitation curve of malate.

FIG. 21: A quantitation curve of N-acetylglucosamine.

FIG. 22: A graphical flow chart that represents the process of performing the extraction and metabolomics as described by the current invention and the subsequent histology of the tissue samples.

FIG. 23: A graph showing the difference between the concentration of the biomarkers uracil, N-acetylaspartate, proline, xanthine, betaine, malate, and N-acetylglucosamine in non-tumor tissue as compared to tumor tissue.

FIG. 24: A graph showing the difference between the concentration of the biomarkers alanine and cysteine in non-tumor tissue as compared to tumor tissue.

### DETAILED DESCRIPTION OF THE INVENTION

The invention is directed to biomarkers for prostate cancer. The invention is also directed to methods of detecting the presence of one or more biomarkers in order to make a diagnosis or prognosis of prostate cancer. The measurement of these markers, alone or in combination, in patient samples, provides information that the diagnostician can correlate with a diagnosis of prostate cancer, risk of developing prostate cancer, and/or prognosis of a subject with prostate cancer. In some embodiments, the biomarkers are betaine, malate, proline, N-acetylaspartate, uracil, xanthine, cysteine, alanine, and N-acetylglucosamine. All nine of these biomarkers can be measured and quantitated at the same time in a single multiplex assay, which could provide very valuable information to the clinician or pathologist. The assay can be varied to quantitate a smaller number of biomarkers if desired.

In one embodiment of the invention, mPREF was used to demonstrate that a subset of metabolites can be quantitated in 18 gauge core needle biopsies of prostate tissue. These metabolites can be used as clinically useful biomarkers. In another embodiment, the quantitation of these biomarkers can be used in the diagnosis or prognosis of prostate cancer. Methods of the present invention can be used independently or in conjunction with currently accepted (or later developed) methods for diagnosing prostate cancer and/or determining the prognosis of a subject with prostate cancer. For example, methods of the present invention may be combined with histology methods.

One aspect of this invention is an assay for quantitating select candidate diagnostic metabolites from cancer needle biopsy extracts using ultra-performance liquid chromatography coupled to a tandem mass spectrometry system (UPLC-MS/MS). In one embodiment, the cancer is prostate cancer. A subset of metabolites from prostate needle biopsies taken from surgical prostatectomy specimens prepared using molecular preservation by extraction and fixation (“mPREF”) were identified as candidate prostate cancer diagnostic biomarkers. (66) In order to determine which metabolites would be the most useful, they were ranked based on the analytical technique that would be required for quantitation (e.g. GC/MS, LC/MS, etc.); the types of verification studies that could be used to confirm that the metabolite was a biomarker for cancer; and the likely ability that the metabolite could be used in a diagnostic assays. Based on this analysis, nine metabolites were identified as potential biomarkers for prostate cancer. In one aspect of the invention, UPLC-MS/MS can be used as the assay platform. However, any LC/MS configuration can be used.

The assay of the present invention was developed and used to quantitate the following biomarkers: betaine, malate, proline, N-acetylaspartate, uracil, xanthine, cysteine, alanine, and N-acetylglucosamine in 29 human prostate biopsy extracts. These involve 8 biochemical groups, which represent pathways of alanine and aspartate metabolism; cysteine, methionine, SAM, and taurine metabolism; glycine, serine, and threonine metabolism; urea cycle, arginine and proline metabolism; aminosugars, glycolysis, pentose metabolism, Krebs cycle, purine (hypoxanthine/inosine containing) and pyrimidine metabolism, respectively.

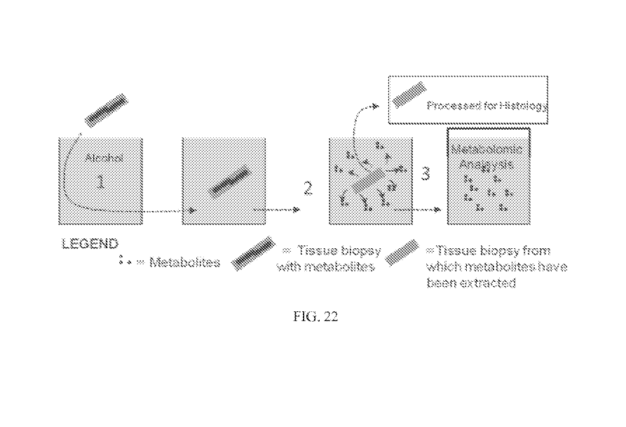

These biomarkers can be used in a multiplexed assay for aiding in the prognosis and diagnosis of cancers, for example prostate cancer. One aspect of the present invention allows for the assay of all nine identified metabolites in a single LC-MS run in a quantitative manner on 18 gauge core needle biopsies. This allows for immediate application in the clinical setting. Then, the tissue can be encased in paraffin and subjected to further processing and histology. A flow chart outlining this procedure is shown in FIG. 22.

One advantage of the current invention is that the biomarkers can be quantitated at the time of a prostate biopsy rather than on prostate tissue samples procured from prostatectomy specimens. Prostatectomy results when the patient and physician have already made a decision to undergo definitive therapy, and have chosen radical prostatectomy. Prognostic information derived from a prostatectomy specimen is not without value, however, the greater value resides in prognostic information contained in the prostate biopsy. The decision point prior to definitive therapy is more crucial, and this is when the patient has been diagnosed with prostate cancer following a biopsy. Prognostic markers useful at this point must therefore be applied to biopsy tissue. The assays of the present invention allow biomarkers to be quantitated from prostate biopsies, and, therefore, can provide information prior to definitive therapy. The treatment options available to patients now include watchful waiting and active surveillance. Active surveillance protocols are problematical since the decision to undergo definitive therapy is substantially influenced by Gleason grading. As described above, this has limitations as interobserver variability occurs. Therefore, quantitative information would be of value to patient and physician.

Quantitation of the biomarkers at the time of prostate biopsy rather than on prostate tissue samples procured from prostatectomy specimens may also be advantageous because it could reduce the effects of ischemia time on metabolites. Metabolite data on human prostate tissue has utilized cryopreserved tissue obtained from prostatectomy specimens. These may be subject to warm ischemia (intraoperative) times of at least 40-60 minutes, prior to any time involved with specimen transport and processing.

Another advantage of the current invention is that it allows for the sampling of the entire prostate when implemented in vivo. Biomarkers can be quantitated in each core regardless of whether histologic tumor is present, and multiple cores with tumor can be sampled. The capability to broadly sample the prostate could be very important since prostate cancer can be heterogeneous.

A further benefit of this invention is that the analysis can be performed on a smaller amount of tissue than the existing diagnostic/prognostic methods. Specifically, this method can be performed on a single 18 gauge needle biopsy that only removes ˜5 mg of tissue. Previous methods for tissue biopsy have required large tissue removal (1 gram or greater) or multiple biopsies of smaller volume (e.g. 18 gauge core biopsies that harvest about 5 mg of tissue). The problem with these two biopsy methods is that they require a significant quantity of tissue to be removed causing greater discomfort and trauma to the subject.

In the methods of the present invention, the fixation of the biomarkers can be performed on a tissue sample and that same tissue sample can then be sent on for further histological evaluation. This is an improvement over the traditional methods of extraction, in which tissue samples were fixed in formalin since formalin is not a suitable extractant for metabolites. Formalin is ineffective in extracting the metabolites and it is reactive so it can alter the metabolite chemistry. Therefore two separate samples (composed of different tissues) had to be harvested using the traditional methods. In the methods of the present invention, a single biological sample (e.g. a tissue biopsy) is contacted with a solvent (e.g. ethanol or methanol) such that the extracted metabolite can be analyzed and the extracted tissue retains its cellular architecture so that it can be subsequently analyzed using standard histological methods (including cytological analysis). The metabolites can then be quantitated and biomarkers may be identified.

A further aspect of this invention is for the analysis to be a multiplexed assay. A multiplex assay is an assay that simultaneously measures multiple biomarkers in a particular sample. The biomarkers can be measured directly from a patient sample with minimal preparation. This allows for a real time assessment of the patient's health in the clinical setting as it relates to the state of the disease. For example, in one embodiment of the invention, samples of prostate cancer biopsy were extracted from various cancer patients. The metabolites were extracted and a series of biomarkers were discovered (betaine, malate, proline, N-acetylaspartate, uracil, xanthine, cysteine, N-acetylglucosamine, and alanine). These biomarkers can be used in a multiplexed analysis of that patient's disease state.

Another advantage of the present invention is that it provides a high throughput method for analyzing biomarkers. Alternatively, using intact tissue, immunohistochemistry (IHC) and fluorescence in situ hybridization (FISH) are used to measure biomarkers in intact tissue. IHC and FISH are technically challenging, generally performed one at a time, and require microscopic interpretation, introducing inter-observer variability. IHC still requires optical detection and interpretation.

Another advantage of the present invention is that the results are quantitative. Metabolite measurements as described herein can be expressed as absolute molar amounts of metabolites. This is a key distinction with IHC results which are notoriously difficult to quantitate.

Betaine, malate, proline, N-acetylaspartate, uracil, xanthine, cysteine, alanine, and N-acetylglucosamine as biomarkers for prostate cancer, as well as methods and uses thereof, are disclosed. These biomarkers are overexpressed in patients with cancer, specifically prostate cancer. These biomarkers can, therefore, be utilized to diagnose patients with cancer, or to diagnose patients at risk for developing cancer. In some embodiments, the invention provides a method of diagnosing prostate cancer in a subject, comprising detecting the differential expression of at least one biomarker in the one or more test samples obtained from the subject, wherein the biomarker is betaine, malate, proline, N-acetylaspartate, uracil, xanthine, cysteine, alanine, and N-acetylglucosamine. In another embodiment, the invention provides a method of determining the prognosis of a subject with prostate cancer, comprising detecting the differential expression of at least one biomarker in the one or more test samples obtained from the subject, wherein the biomarker is betaine, malate, proline, N-acetylaspartate, uracil, xanthine, cysteine, alanine, and N-acetylglucosamine.

In one embodiment of the present invention, a method of diagnosing cancer, specifically prostate cancer, or risk for developing cancer in a subject is provided. This method comprises the steps of (a) providing a biological sample from the subject; (b) contacting the biological sample with an extraction reagent capable of extracting the cancer biomarkers comprising betaine, malate, proline, N-acetylaspartate, uracil, xanthine, cysteine, alanine, and N-acetylglucosamine; (c) determining the amount of the biomarkers; and (d) correlating the amount biomakers to a prostate cancer diagnosis.

The amount of cancer biomarkers (i.e., betaine, malate, proline, N-acetylaspartate, uracil, xanthine, cysteine, alanine, and N-acetylglucosamine) in normal biological samples can be assessed in a variety of ways as described herein. In one embodiment, the normal or control amount of biomarkers can be determined by assessing the amount of betaine, malate, proline, N-acetylaspartate, uracil, xanthine, cysteine, alanine, and N-acetylglucosamine, in one or more samples obtained from one or more individuals without cancer.

Using the methods of the invention, levels of prostate cancer biomarkers (i.e., betaine, malate, proline, N-acetylaspartate, uracil, xanthine, cysteine, alanine, and N-acetylglucosamine) are determined in a biological sample from a subject suspected of having prostate cancer and in one or more comparable biological samples from normal or healthy subjects (i.e., control samples). A level of prostate cancer biomarker (i.e., betaine, malate, proline, N-acetylaspartate, uracil, xanthine, cysteine, alanine, and N-acetylglucosamine) detected in a biological sample from a subject suspected of having prostate cancer that is higher than the prostate cancer biomarker level detected in a comparable biological sample from a normal or healthy subject, indicates that the subject suspected of having prostate cancer has or is likely to have prostate cancer.

The biomarkers of the present invention can also be quantitated and correlated with various stages of a disease. For example, the biomarkers can be used to determine whether a subject has stage pT2 disease or pT3 disease of prostate cancer. At present, there is no way of confidently distinguishing pT2 from pT3 disease unless a prostatectomy is performed. Using the methods of the invention, levels of prostate cancer biomarkers (i.e., betaine, malate, proline, N-acetylaspartate, uracil, xanthine, cysteine, alanine, and N-acetylglucosamine) could be determined in a biological sample from a subject suspected of having prostate cancer and in one or more comparable biological samples from subjects with different stages (i.e., pT2 or pT3) of the disease. A level of prostate cancer biomarker (i.e., betaine, malate, proline, N-acetylaspartate, uracil, xanthine, cysteine, alanine, and N-acetylglucosamine) detected in a biological sample from a subject suspected of having prostate cancer that is comparable to prostate cancer biomarker level detected in a comparable biological sample from a subject with a pT2 or pT3 stage of the disease, would be instructive as to what stage of prostate cancer is present in the tested subject.

In other aspects of the invention, the quantitation of biomarkers in conjunction with mPREF techniques can be done for biomarkers other than those for prostate cancer. The quantitation methods of the current invention are applicable to any tissue biopsy and any disease state, and are not limited to a single organ site. Other possible applications of the methods of the present invention include inflammatory skin diseases, diabetes, allografts for analysis of rejection, muscle and nerve biopsies, and cytologic specimens such as fine needle aspirates and smears.

In accordance with the invention, at least one biomarker may be detected. It is to be understood, and is described herein, that one or more biomarkers may be detected and subsequently analyzed, including several or all of the biomarkers identified.

It is to be understood that normal histology methods can also be used in conjunction with the methods of the invention. This is accomplished by imaging the slides to estimate tumor volume. The methods may combine automated feature recognition with manual (pathologist-assisted) feature recognition and assignment to optimize workflow. One aspect of the invention is to produce a high throughput system that can reasonably approximate the surface areas including 1) Total surface area of specimen, 2) Surface area of benign epithelium, 3) Surface area of tumor epithelium, and 4) Surface area of stroma. This information can then be used with computer-assisted software to efficiently correlate metabolite data with histology.

The methods of the invention can also be used in conjunction with a graphical user interface (“GUI”) that displays normalized metabolite values with standard text based pathology biopsy reports in a visually ergonomic fashion. Specifically, the invention can be used to display a pathological report that displays relative risk with each positive core on the “front sheet” readily visible to the clinician (urologist; oncologist) end user. A more detailed display with quantification would be on a “back sheet”. Each specimen received (each core) in the laboratory requires generation of a pathology report which conveys in two or three lines of text, the diagnosis, Gleason score and grade, an estimate of the percent of biopsy involved by tumor, and the number of cores involved by tumor. The normalized metabolite data can be combined with the traditional pathology report. It can be read quickly, readily interpreted, and can be expressed as a relative risk of T3 vs. T2 disease etc. Recent studies have shown that many methods of digital image processing can be used for prostate imaging (63, 64, 65). These include computer-assisted tools and software for processing pathological prostate images for automatic classification and accurate grading of prostate tissues.

Although mPREF techniques are described herein, the biomarkers of the present invention may be detected from any biological sample from a subject. The biological sample may be a biological fluid such as whole blood or serum. The biological sample may also be from tissue such as prostate tissue. Other examples of tissue specimen useful to practice the general methods of the present invention include samples taken from the prostate, central nervous system, bone, breast tissue, renal tissue, endometrium, head/neck, gall bladder, parotid tissue, brain, pituitary gland, kidney tissue, muscle, esophagus, stomach, small intestine, colon, urethra, liver, spleen, pancreas, thyroid tissue, heart, lung, bladder, adipose tissue, lymph node tissue, adrenal tissue, testis tissue, tonsils, and thymus.

Biomarkers of the present invention may also be detected from biological fluid such as whole blood, serum, plasma, urine, tears, mucus ascites fluid, oral fluid, saliva, semen, seminal fluid, mucus, stool, sputum, cerebrospinal fluid, bone marrow, lymph, and fetal fluid. The biological fluid samples may include cells, proteins, or membrane extracts of cells.

“Subject” includes living and dead organisms. Examples of subjects include mammals, e.g., humans, dogs, cows, horses, pigs, sheep, goats, cats, mice, rabbits, rats, and transgenic nonhuman animals. Most preferably the subject is a human.

The biomarkers of this invention can be isolated and purified from biological fluids, such as urine or serum. They can be isolated by any method known in the art, based on their mass, their binding characteristics and their identity as betaine, malate, proline, N-acetylaspartate, uracil, xanthine, cysteine, alanine, and N-acetylglucosamine. For example, a biological sample comprising the biomarkers can be subject to chromatographic fractionation and subject to further separation.

“Purified” means substantially pure and refers to biomarkers that are substantially free of other proteins, lipids, carbohydrates or other materials with which they are naturally associated.

Monitoring the Effect of an Anti-Prostate Cancer Drug or Therapy Administered to a Subject with Prostate Cancer

In another embodiment of the present invention, a subject's prostate cancer status is determined as part of monitoring the effect of an anti-prostate cancer drug or a therapy administered to the subject diagnosed with prostate cancer. The effect of an anti-prostate cancer drug or a therapy administered to a subject with prostate cancer may include the worsening or improvement of prostate cancer processes.

Using the methods of the invention, levels of betaine, malate, proline, N-acetylaspartate, uracil, xanthine, cysteine, alanine, or N-acetylglucosamine are determined in a biological sample from a subject at various times of having been given an anti-prostate cancer drug or a therapy. A prostate cancer biomarker level detected in a biological sample from a subject at a first time (t1; e.g., before giving an anti-prostate cancer drug or a therapy) that is higher than the prostate cancer biomarker level detected in a comparable biological sample from the same subject taken at a second time (t2; e.g., after giving an anti-prostate cancer drug or therapy), indicates that the cancer in the subject is regressing. Likewise, a higher prostate cancer biomarker level at a second time compared to a prostate cancer biomarker level at a first time, indicates that the cancer in the subject is progressing.

In another embodiment, this method involves measuring one or more prostate cancer biomarkers, one of which may be betaine, malate, proline, N-acetylaspartate, uracil, xanthine, cysteine, alanine, or N-acetylglucosamine, in a subject at least at two different time points, e.g., a first time and a second time, and comparing the change in amounts, if any. The effect of the anti-prostate cancer drug or therapy on the progression or regression of the cancer is determined based on these comparisons. Thus, this method is useful for determining the response to treatment. If a treatment is effective, then the prostate cancer biomarker will trend toward normal, while if treatment is ineffective, the prostate cancer biomarker will trend toward disease indications.

In another embodiment, the method involves measuring one or more metastases of prostate cancer biomarkers, one of which may be betaine, malate, proline, N-acetylaspartate, uracil, xanthine, cysteine, alanine, or N-acetylglucosamine, in a subject at least at two different time points, e.g., a first time and a second time, and comparing the change in amounts, if any. This is done to assess the state of the disease, the progression of the disease and the likelihood of response to a treatment.

### Kits for Diagnosing and Assessing Prognosis of Prostate Cancer

The methods, alone or in combination of the present invention, may be provided in the form of a kit. Kits may further comprise appropriate controls and/or detection reagents. In an embodiment, the kit may include tools and reagents for the analysis of a tissue sample or biopsy. The kit may comprise a sample collection element and a tool for placing the biopsy or tissue sample into the collection element. The collection element may contain extraction solvent, a tool to retrieve the tissue following incubation, and a tool to place the collected tissue sample into a collection receptacle for histological analyses. For example, the kit may comprise a sample collection element, an extraction solvent, a tissue retrieval element, a retrieved tissue collection receptacle, sample labels, sample barcodes, and instruction protocol. The instruction protocol may be provided as a printed form or booklet or on an electronic medium, such as, for example, a computer disk or other computer readable medium.

### Examples

The following examples are presented for the purpose of illustration only and are not intended to be limiting.

Methods for quantitating potential prostate cancer biomarkers are provided below.

### Materials and Methods

Reference standards and stable isotope-labeled standards were purchased from Sigma-Aldrich (St. Louis, Mo.) including betaine, malate, proline, N-acetylaspartate, uracil, xanthine, cysteine, and alanine. Stable isotope-labeled chemicals including betaine-trimethyl-d5 hydrochloride, xanthine 1,3-N15, cysteine C13, malate-2,3,3-d3, and L-proline 2,5,5-d3 were obtained from Cambridge Isotope Laboratories Inc. (Andover, Mass.) and uracil-d4, N-acetyl-aspartate 2,3,3-d3, and alanine-C13 were from C/D/N ISOTOPES INC. (Quebec, Canada). Chemicals including methanol (Optima LC-MS), acetonitrile (Optima LC-MS), and formic acid (Optima LC-MS) were purchased from Thermo Fisher Scientific (FairLawn, N.J.). Ammonium acetate and acetic acid (Glacial) were purchased from Sigma-Aldrich. Sodium formate solution (0.05 M NaOH+0.5% formic acid in 90:10 2-propanol: water, Waters Corp., Milford, Mass.) was used for instrument tuning and calibration. Ultrapure water was produced by Mill-Q Reference system equipped with a LC-MS Pak filter (Millipore, Billerica, Mass.).

### I. Quantitation Experiments

A total of 29 extract samples were obtained from surgical prostatectomy specimens of patients who elected prostatectomy as a primary treatment. The biopsies were obtained ex vivo and were stored at 4° C. until sample preparation and analysis. A pooled quality control sample was prepared by mixing each aliquot of the test samples for assay validation and concentration estimation.

### Sample Preparation

Each 1 mL of sample was transferred to a 5-mL glass vial and dried under gentle nitrogen at room temperature (Glas-Col Nitrogen Evaporator System, Terre Haute, Ind.). The residue was reconstituted with 100 μL of acetonitrile plus 100 μL of water. The mixture was centrifuged at 14,500 rpm for 20 min (Microfuge 22R centrifuge, Beckman Coulter, Inc., Atlanta, Ga.). A 150-1 μL aliquot of the resulting supernatant was transferred to a 2-mL glass sample vial and analyzed on a UPLC-MS/MS system (Waters Corp., Milford, Mass.).

### Quantitation Curve

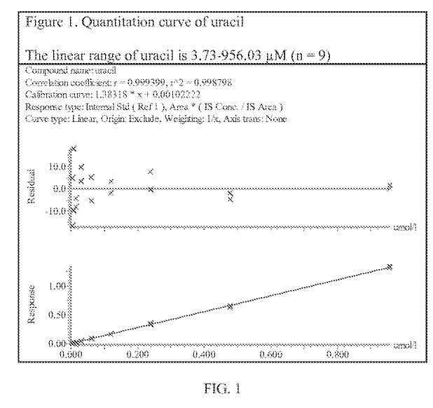

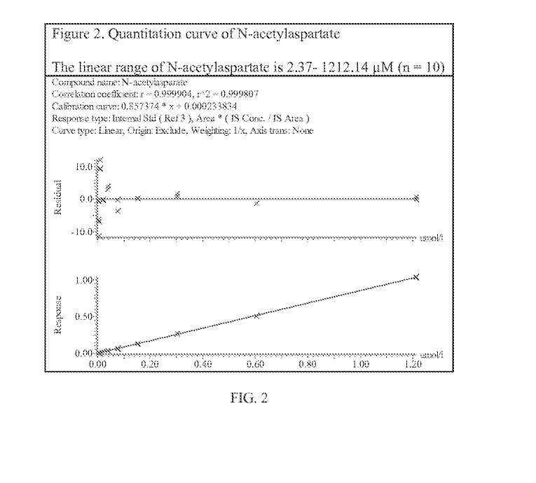

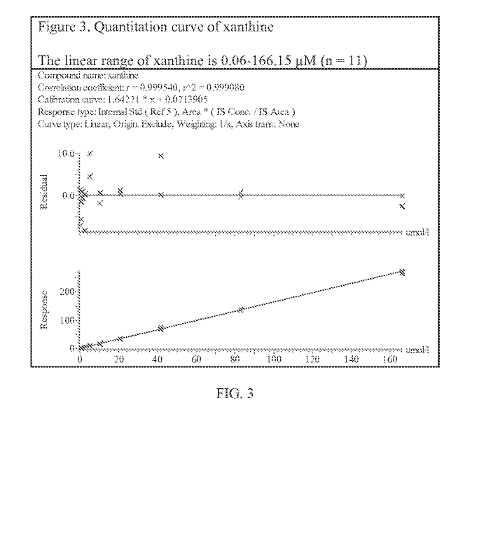

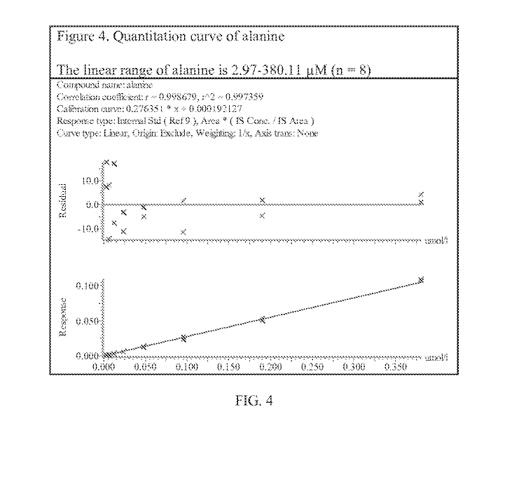

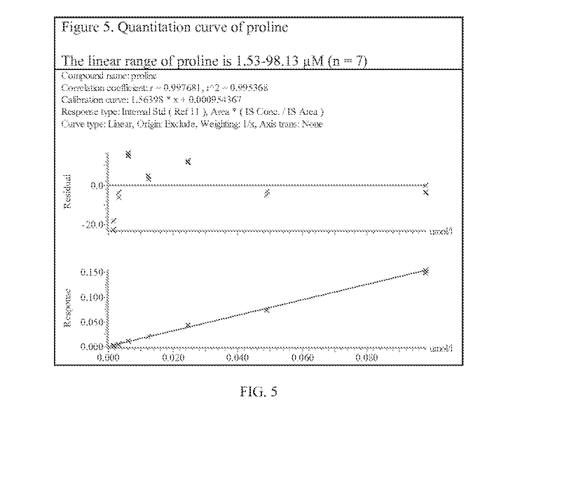

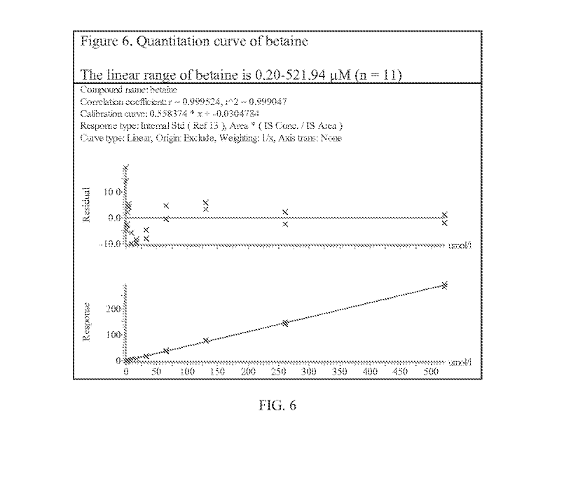

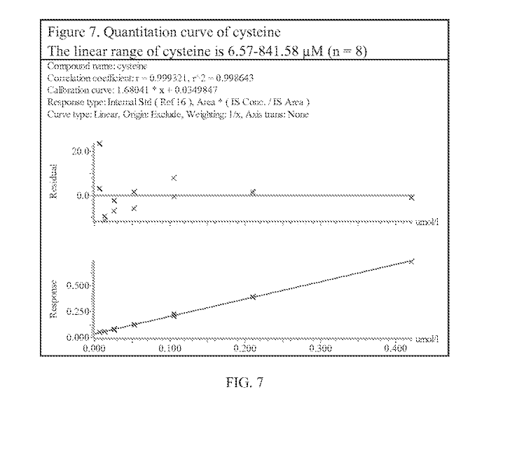

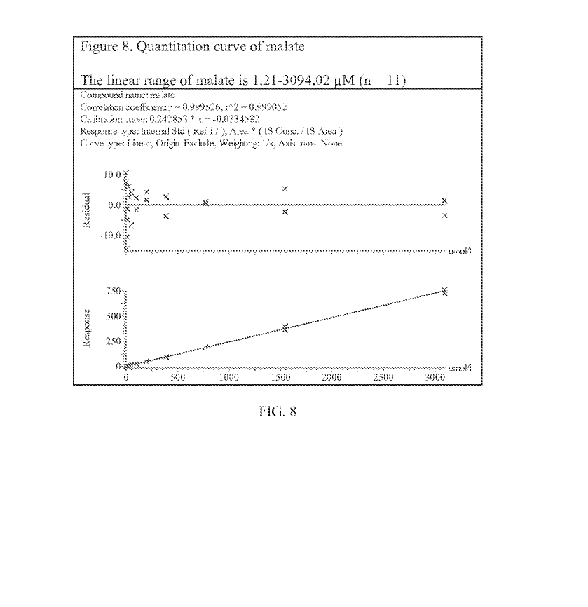

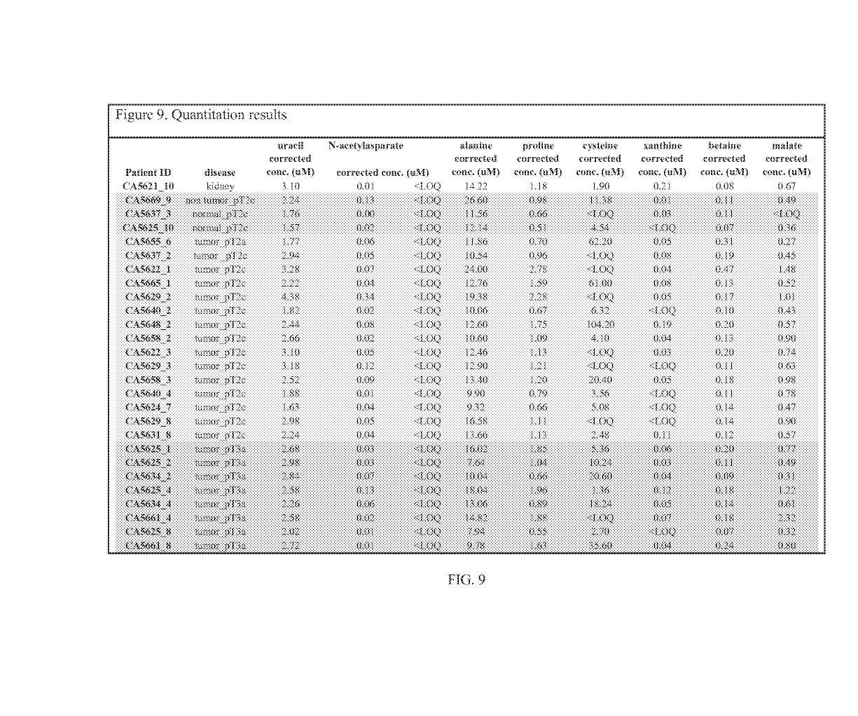

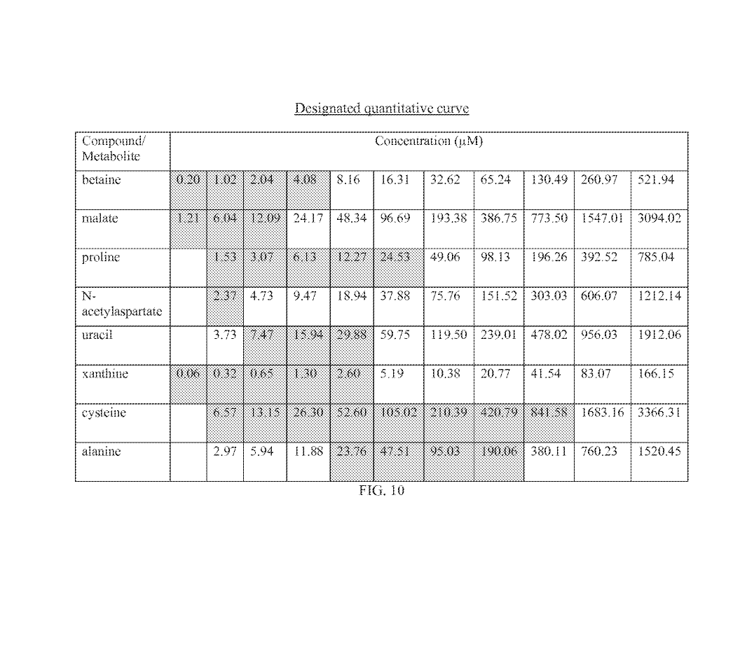

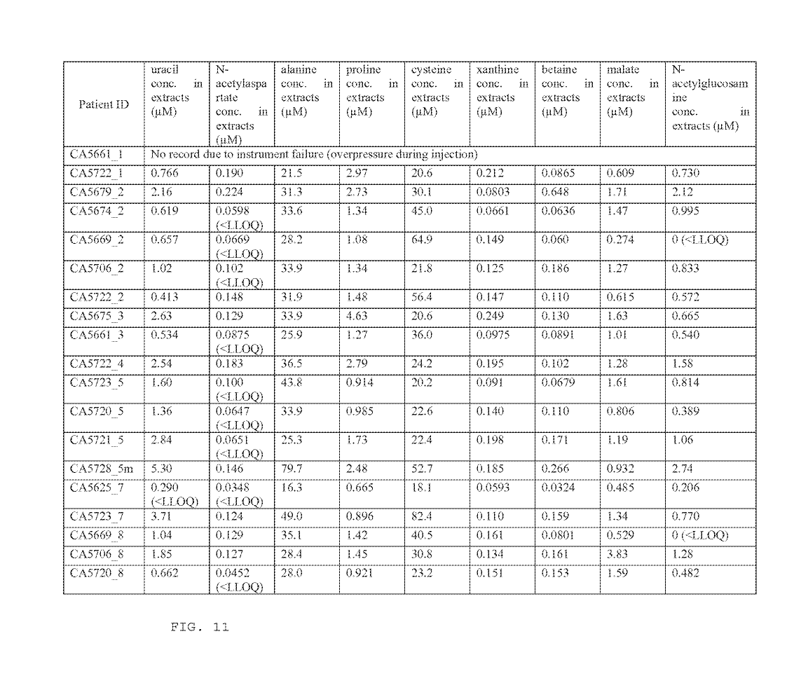

Both reference standards and stable isotope-labeled standards were dissolved in appropriate solvents as indicated below. As the quantitation curve range is different for each compound and the concentration of each metabolite present in real samples varies, the pooled sample was used to estimate the quantitation range. The designated concentrations for each compound/metabolite were obtained. The calibration/linearity equation and corresponding regression coefficients (R2) were calculated using the QuanLynx Application Manager (Waters Corp., Milford, Mass.) and the limit of quantitation was defined as the lowest concentration in the calibration curve in this study. The calibration curves generated in these experiments for biomarkers cysteine, malate, uracil, N-acetylaspartate, xanthine, alanine, betaine, and proline are shown in FIGS. 1 to 8. FIG. 9 contains all of the actual biomarker measurements from the various tissue samples in this set of experiments. FIG. 10 demonstrates that the biomarkers of the present invention are capable of being quantitated because the measured value of a particular biomarker in the tissue fell within the range of the calibration curve for that biomarker. In the figure, the boxes in gray indicate the range on the calibration curve where the quantity of biomarker in the tissue samples fell. The numerical values in the figure are the actual values of the calibration curves.

Several mobile phases were compared during these experiments including I) A: acetonitrile (0.2% formic acid, pH=3), and B: water (0.2% formic acid, pH=3); II) A: acetonitrile, and B: water; III) A: acetonitrile (5 mM ammonium acetate, pH=5.5), and B: water (5 mM ammonium acetate, pH=6); IV) A: acetonitrile (5 mM ammonium acetate, pH=3.8), and B: water (5 mM ammonium acetate, pH=4); V) A: acetonitrile (5 mM ammonium acetate, pH=3), and B: water (5 mM ammonium acetate, pH=3). Based on chromatographic performance and overall sensitivity, the mobile phase I was finally selected. The elution gradient was optimized with the goal of separating as many as possible of the targeted compounds without significantly increasing analytical time.

Different reconstitution solvents were compared to maximize the extraction efficiency without compromising the chromatographic performance. A total of five representative solvents were used including I) acetonitrile/methanol=75:25 (v/v); II) acetonitrile/water=80:20 (v/v); III) acetonitrile/water=65:35 (v/v); IV) acetonitrile/water=50:50 (v/v); V) water. The optimum dilution and reconstitution solvent was acetonitrile/water=50:50 (v/v) in terms of peak shapes and recovery. A maximal and consistent recovery was achieved by a two-step reconstitution with 100 μL of acetonitrile followed by 100 μL of water.

A UPLC-MS/MS system (ACQUITY UPLC-Quattro Premier XE MS, Waters Corp., Milford, Mass.) was used. The system was operated in electrospray ionization (ESI) positive mode. The optimized instrument settings are briefly described in Table 1a-b. (can probably leave as table but have to renumber) Gradient solvent B=0.2% formic acid in water and gradient solvent A=0.2% formic acid in acetonitrile.

Tables 1a and 1b below show the instrument settings for this set of experiments:

TABLE 1a
UPLC-MS/MS instrument settings
UPLC

| Column | ACQUITY UPLC BEH HILIC 1.7 μM |
|---|---|
| | VanGuard pre-column (2.1 × 5 mm) and |
| | ACQUITY UPLC BEH HILIC 1.7 μM |
| | analytical column (2.1 × 100 mm) |
| Column Temp (° C.) | 40 ± 0.5 |
| Sample Manager | 4 ± 0.5 |
| Temp (° C.) | |
| Gradient Conditions | 0-1 min (5% B), 1-2.5 min (5-10% B), |
| | 2.5-5 min (10-20% B), 5-7 min (20- |
| | 100% B), 7-8 min (100% B), 8-8.2 min |
| | (100-5% B), 8.2-9 min (5% B). |
| Flow Rate (mL/min) | 0.40 |

Quattro XE Premier MS

| | |
|---|---|
| Capillary (kV) | 4.0 |
| Sampling Cone (V) | See Table 1b for details |
| Collision Energy | See Table 1b for details |
| Extraction Cone (V) | 4.0 |
| Source Temp (° C.) | 120 |
| Desolvation Temp (° C.) | 350 |
| Desolvation Gas Flow | 1000 |
| (L/Hr) | |
| Cone Gas (L/Hr) | 50 |

TABLE 1b
MS/MS parameters for compound detection

| | Multiple reaction | | |
|---|---|---|---|
| | monitor | Cone | Collision |
| Compound | (MRM) transition | voltage | energy |
| alanine | 90 > 44 | 30 | 10 |
| alanine-C13 | 91 > 45 | 25 | 10 |
| uracil | 113 > 96 | 30 | 15 |
| uracil-d4 | 115 > 98 | 35 | 20 |
| proline | 116 > 70 | 35 | 10 |
| L-proline-2,5,5-d3 | 119 > 73 | 40 | 15 |
| cysteine | 122 > 76 | 20 | 10 |
| Cysteine-C13 | 123 > 16 | 25 | 15 |
| betaine | 118 > 59 | 20 | 15 |
| betaine-trimethyl-d5 | 127 > 68 | 35 | 15 |
| hydrochloride | | | |
| xanthine | 153 > 110 | 30 | 15 |
| xanthine 1,3-N15 | 155 > 111 | 35 | 20 |
| N-acetylaspartate | 176 > 134 | 20 | 10 |
| N-acetyl-aspartate 2,3,3-d3 | 179 > 137 | 20 | 10 |
| malate | 133 > 71^{a} | 25 | 15 |
| malate 2,3,3-d3 | 136 > 117^{a} | 25 | 15 |

^{a}These compounds were detected in negative mode.

Using UPLC-MS/MS with MRM mode, the following metabolites were within the concentration range: betaine, malate, proline, uracil, xanthine, cysteine, and alanine. Although the concentration of N-acetylaspartate in the test sample was on the edge of the LOQ, it is still a viable biomarker because further refinement of the quantitation methods cause it to be within the linear range of the curve (see FIG. 10).

This first set of experiments (above) was used to determine which metabolites could be quantitated such that they could be viable biomarkers for prostate cancer. The next set of experiments (below) confirmed these metabolites as biomarkers for prostate cancer.

### II. Identification of Prostate Cancer Biomarkers

Single core needle biopsies obtained ex vivo from surgical prostatectomy specimens were immediately placed in 80% aqueous alcohol and transferred to formalin after 12-24 hours. The alcohol was retained, dried down and the residue reconstituted with 95 μL of acetonitrile plus 95 μL of water. Histology was performed on exactly the same tissue.

### Standards and Solvents

Reference standards and stable isotope-labeled standards were purchased from Sigma-Aldrich (St. Louis, Mo.) including betaine, malate, proline, N-acetylaspartate, N-acetylglucosamine, uracil, xanthine, cysteine, alanine. Stable isotope-labeled chemicals including glucosamine C13 dydrochloride, betaine-trimethyl-d5 hydrochloride, xanthine 1,3-N15, cysteine C13, malate-2,3,3-d3, and L-proline 2,5,5-d3 were obtained from Cambridge Isotope Laboratories Inc. (Andover, Mass.) and uracil-d4, N-acetyl-aspartate 2,3,3-d3, and alanine-C13 were from C/D/N ISOTOPES INC. (Quebec, Canada). Chemicals including methanol (Optima LC-MS), acetonitrile (Optima LC-MS) and formic acid (Optima LC-MS) were purchased from Thermo Fisher Scientific (FairLawn, N.J.). Ammonium acetate and acetic acid (Glacial) were purchased from Sigma-Aldrich. Sodium formate solution (0.05 M NaOH+0.5% formic acid in 90:10 2-propanol:water, Waters Corp., Milford, Mass.) was used for instrument tuning and calibration. Ultrapure water was produced by a Mill-Q Reference system equipped with a LC-MS Pak filter (Millipore, Billerica, Mass.).

### Source of Samples

A total of 30 study samples extracted from prostate needle biopsies were provided and stored at 4° C. prior to sample preparation and analysis. The sample (CA5661_{—}1) was not analyzed due to instrument failure (over-pressurization) during injection. The actual number of samples analyzed was 29. The tissue samples from 12 patients used to quantitate the biomarkers were as follows: 15 Tumor samples (1 sample pT3a and 14 samples pT2) and 14 Paired adjacent non-tumor sample.

### Sample Preparation

Each 1.9 mL of sample was transferred to a 5 mL glass vial and dried under a gentle nitrogen flow at room temperature (Glas-Col Nitrogen Evaporator System, Terre Haute, Ind.). The residue was reconstituted with 95 μL of acetonitrile plus 95 μL of water. The mixture was centrifuged at 14,500 rpm for 20 min (Microfuge 22R centrifuge, Beckman Coulter, Inc., Atlanta, Ga.). A 150 μL aliquot of the resulting supernatant was transferred to 2 mL glass sample vial and analyzed on a UPLC-MS/MS system (Waters Corp., Milford, Mass.).

### Quantitation Curve

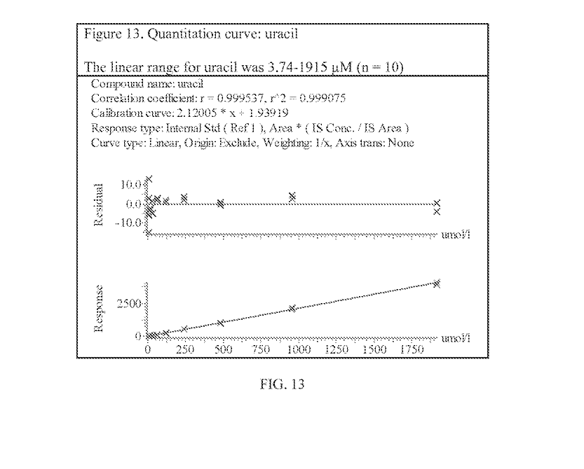

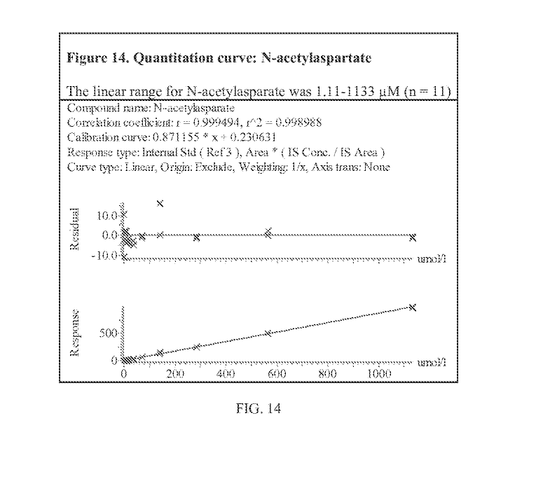

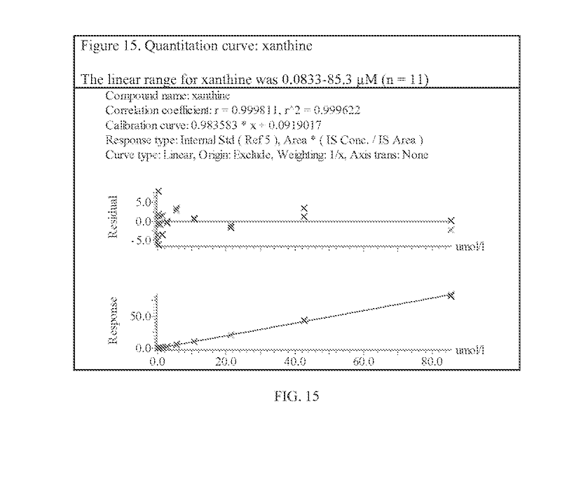

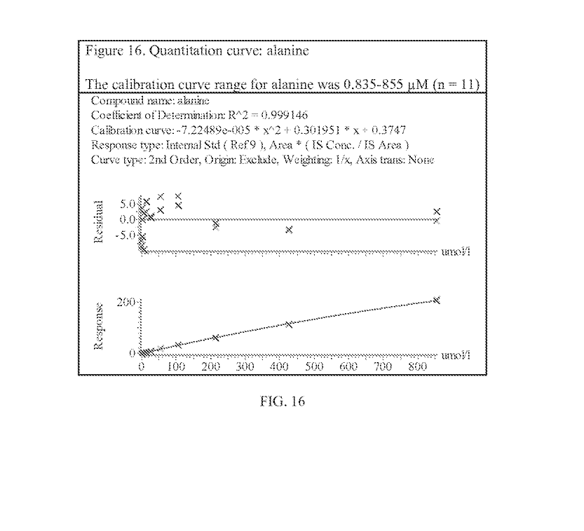

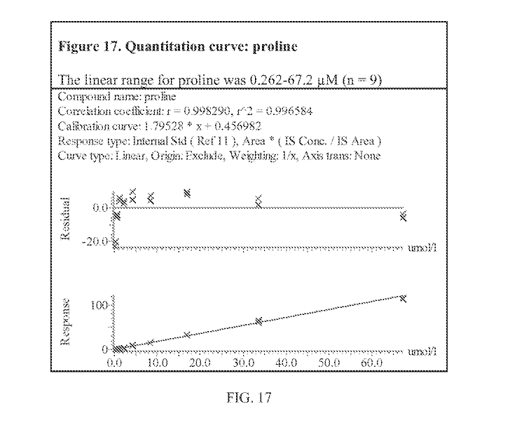

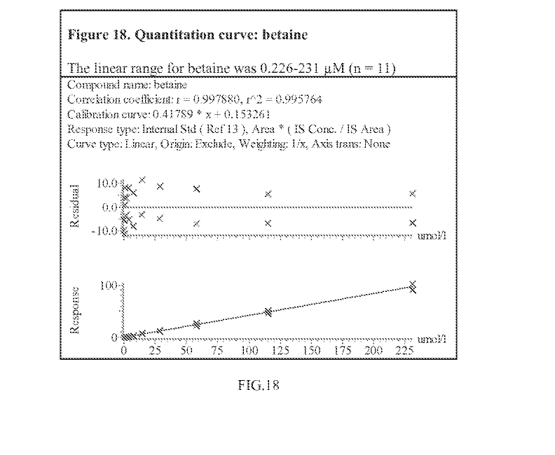

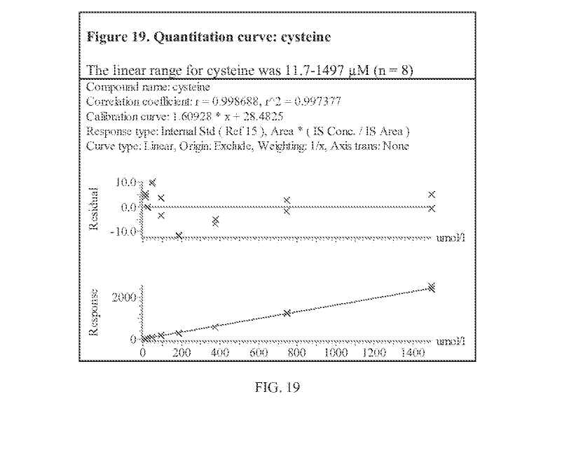

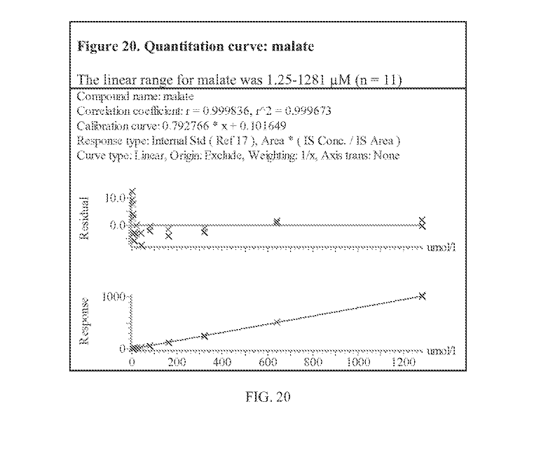

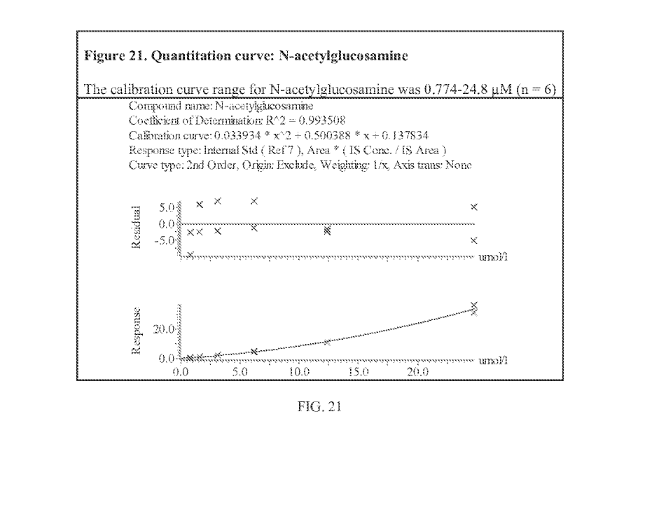

Both reference standards and stable isotope-labeled standards were dissolved in appropriate solvents (methanol or water) based on their solubility. The stable isotope-labeled standards were used as internal standards for their corresponding analytes, and thus were used to compensate for possible variations during sample preparation, injection, chromatography, matrix effects, etc. The quantitation curve solutions were prepared by mixing each aliquot of reference standard stock solution followed by a series of dilution with a mixture of methanol and water (50:50, v/v). The designated concentrations for each compound were obtained. The calibration equation and corresponding regression coefficients (R2) were calculated using the QuanLynx Application Manager (Waters Corp., Milford, Mass.) and the limit of quantitation was defined as the lowest concentration in the calibration curve. These curves are shown in FIGS. 13-21.

mPREF samples including tumor/non-tumor were analyzed using the developed assay method described above and the concentration ranges obtained. These metabolites were assayed on a single LC/MS run.

Tables 2a and 2b below show the instrument settings for this set of experiments:

TABLE 2a
UPLC-MS/MS instrument settings
UPLC

| Column | ACQUITY UPLC BEH HILIC 1.7 μM |
|---|---|
| | VanGuard pre-column (2.1 × 5 mm) and |
| | ACQUITY UPLC BEH HILIC 1.7 μM |
| | analytical column (2.1 × 100 mm) |
| Column Temp (° C.) | 40 ± 0.5 |
| Sample Manager | 4 ± 0.5 |
| Temp (° C.) | |
| Gradient Conditions | 0-1 min (5% B), 1-2.5 min (5-10% B), |
| | 2.5-5 min (10-20% B), 5-7 min (20- |
| | 100% B), 7-8 min (100% B), 8-8.2 min |
| | (100-5% B), 8.2-9 min (5% B). |
| Flow Rate (mL/min) | 0.40 |

Quattro XE Premier MS

| | |
|---|---|
| Capillary (kV) | 4.0 |
| Sampling Cone (V) | See Table 2b for details |
| Collision Energy | See Table 2b for details |
| Extraction Cone (V) | 4.0 |
| Source Temp (° C.) | 120 |
| Desolvation Temp (° C.) | 350 |
| Desolvation Gas Flow | 1000 |
| (L/Hr) | |
| Cone Gas (L/Hr) | 50 |

TABLE 2b
MS/MS parameters for compound detection

| | Multiple reaction | Cone | |
|---|---|---|---|
| | monitor | voltage | Collision |
| Compound | (MRM) transition | (V) | energy (V) |
| alanine | 90 > 44 | 30 | 10 |
| alanine-C13 | 91 > 45 | 25 | 10 |
| uracil | 113 > 96 | 30 | 15 |
| uracil-d4 | 115 > 98 | 35 | 20 |
| proline | 116 > 70 | 35 | 10 |
| L-proline-2,5,5-d3 | 119 > 73 | 40 | 15 |
| cysteine | 122 > 76 | 20 | 10 |
| cysteine-C13 | 123 > 76 | 25 | 15 |
| betaine | 118 > 59 | 20 | 15 |
| betaine-trimethyl-d5 | 127 > 68 | 35 | 15 |
| hydrochloride | | | |
| xanthine | 153 > 110 | 30 | 15 |
| xanthine1,3-N15 | 155 > 111 | 35 | 20 |
| N-acetylaspartate | 176 > 134 | 20 | 10 |
| N-acetyl-aspartate 2,3,3-d3 | 179 > 137 | 20 | 10 |
| malate^{a} | 133 > 71 | 25 | 15 |
| malate 2,3,3-d3^{a} | 136 > 117 | 25 | 15 |
| N-acetylglucosamine | 222 > 138 | 20 | 15 |
| glucosamine-C13 | 181 > 73 | 30 | 15 |
| dydrochloride | | | |

^{a}These compounds were detected in negative mode.

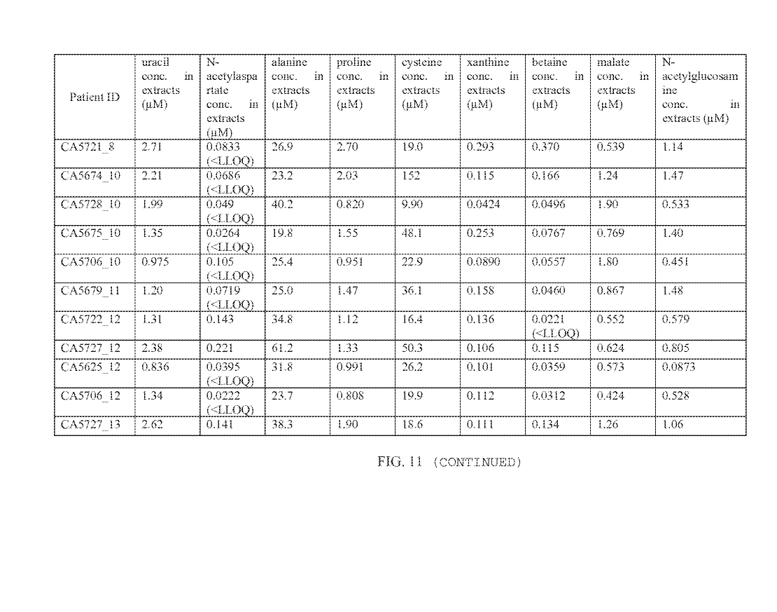

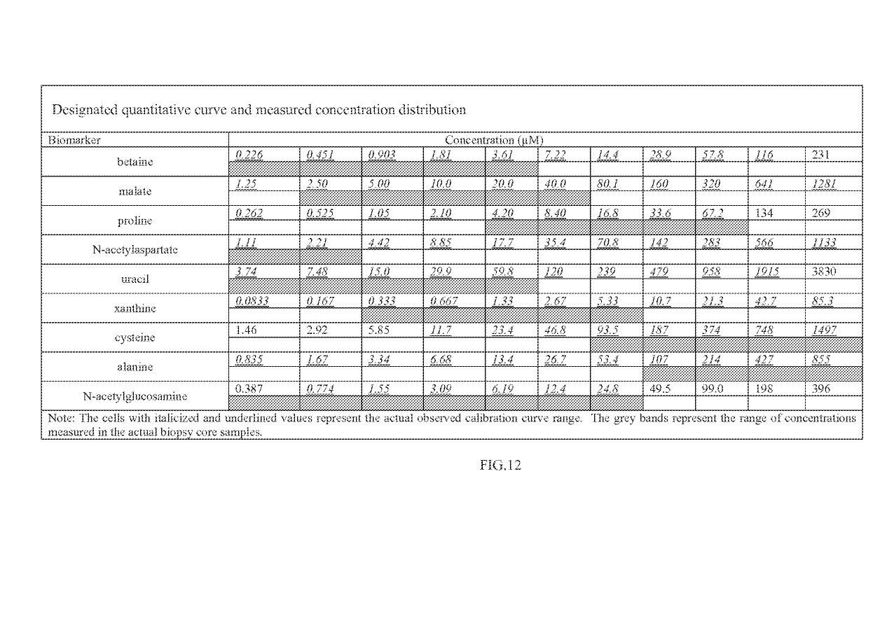

FIGS. 13 to 21 are the calibration curves for each of the biomarkers analyzed in these experiments. FIG. 11 contains the quantitation data for the biomarkers of the present invention in this set of experiments. FIG. 12 demonstrates that the quantitation range for each biomarker (represented by the grey band) falls within the linear portion of the calibration curve for the biomarkers. Therefore, each of these biomarkers can be quantitated in an 18 gauge core biopsy of prostate tissue.

### III. Normalization of Biomarkers to Tumor Surface Area

Morphometric determination of the amount of tumor, non-tumor, glands and stroma in a given biopsy was used to determine that the biomarkers of the present invention correspond with the amount of tumor present in a biopsy. The data was normalized to the total biopsy surface area for each biopsy core. Biopsy surface area was obtained by scanning glass slides on a high resolution flatbed scanner. Dark pixels were converted to surface area using the program “ImageJ”. The resulting surface area was then used to convert data to μM/cm2. The μM/cm2 values for samples without tumor and with tumor are provided below in Table 3.

TABLE 3

| | No | | No | | Tumor | | |
|---|---|---|---|---|---|---|---|
| | Tumor | Tumor | Tumor | Tumor | Corrected | Lo Std | Hi Std |
| Biomarker | uM | uM | uM/cm2 | uM/cm2 | uM | uM | uM |
| betaine | 0.079 | 0.189 | 2.041 | 4.712 | 0.46 | 0.226 | 231 |
| malate | 1.017 | 1.248 | 30.233 | 33.92 | 3.47 | 1.25 | 1281 |
| proline | 1.472 | 1.763 | 42.943 | 47.543 | 4.99 | 0.262 | 269 |
| N-acetylaspartate | 0.171 | 0.153 | 2.794 | 2.992 | 0.312 | 1.11 | 1133 |
| uracil | 1.443 | 2.030 | 41.127 | 50.555 | 5.15 | 3.74 | 3830 |
| xanthine | 0.127 | 0.154 | 3.898 | 4.377 | 0.766 | 0.0833 | 85.3 |
| cysteine | 28.39 | 44.71 | 877.901 | 1209.76 | 127.94 | 1.46 | 1497 |
| alanine | 31.51 | 35.27 | 948.749 | 927.435 | 113.7 | 0.835 | 855 |
| N- | 0.698 | 1.195 | 20.859 | 28.970 | 2.57 | 0.387 | 396 |
| acetylglucosamine | | | | | | | |

In table 3 above, No Tumor uM is the raw data; uM/Cm2 is the data normalized to surface area of the biopsy core; Lo Std/Hi Std are the low and high end of the standard curves. The calculation of the “Tumor Corrected” data is described below.

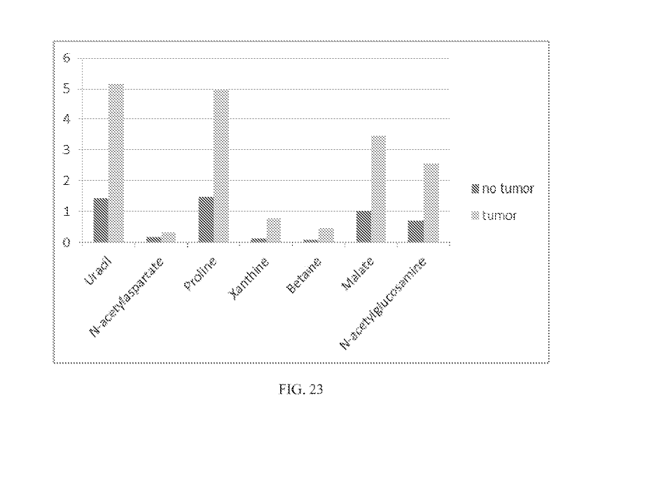

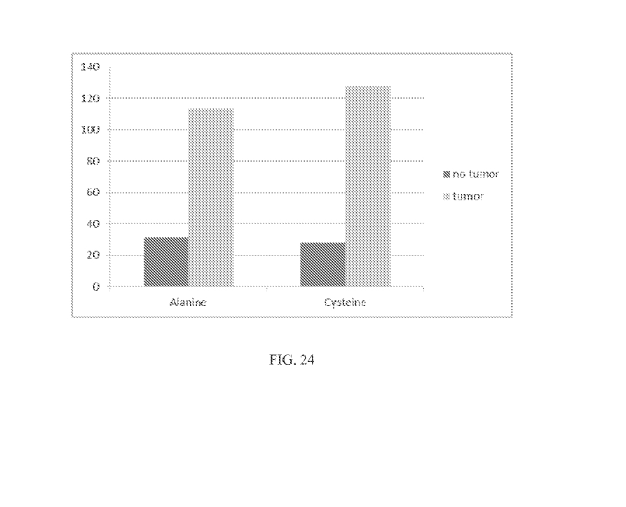

The data was also normalized based upon percent surface area of tumor. Percent tumor was assessed by microscopic examination of the biopsies by a pathologist. Raw data for each biomarker was converted to the equivalent of 100% tumor by expressing percent in decimal form and multiplying by the appropriate factor to equal one (e.g. 10%=0.1; 0.1×10=1). This is referred to as “tumor corrected” in Table 3. The no tumor values were then compared to the tumor corrected values to demonstrate the difference between the amount of the biomarker in normal tissue and the amount in tumorous tissue. FIG. 23 shows the comparison of the uracil, N-acetylaspartate, proline, xanthine, betaine, malate, and N-acetylglucosamine biomarkers in normal tissue (black) versus tumor tissue (gray). FIG. 24 shows the comparison of the cysteine and alanine levels in normal tissue (black) versus tumor (gray). These data indicate that the biomarkers of the present invention can be used to quantitatively determine the amount of cancer tissue in a particular prostate biopsy. Therefore, these biomarkers can be used to detect prostate cancer, determine prostate cancer prognosis, monitor the treatment of the disease, and screen possible new treatments for prostate cancer.

While the invention has been illustrated and described in the figures and foregoing description, the same is to be considered as illustrative and not restrictive in character, it being understood that only the preferred embodiments have been shown and described and that all changes and modifications that come within the spirit of the invention are desired to be protected. In addition, all references and patents cited herein are indicative of the level of skill in the art and hereby incorporated by reference in their entirety.

### REFERENCES

1. Ries L A G MD, Krapcho M, Stinchcomb D G, Howlader N, Horner M J, Mariotto A, Miller B A, Feuer E J, Altekruse S F, Lewis D R, Clegg L, Eisner M P, Reichman M, Edwards B K (eds). SEER Cancer Statistics Review, 1975-2005, National Cancer Institute Bethesda, Md. 2008: http://seer.cancer.gov/csr/1975.sub.—2005/, based on November 2007 SEER data submission, posted to the SEER web site.
2. Nelen V. Epidemiology of prostate cancer. Recent Results in Cancer Research 2007; 175: 1-8.
3. Thompson I M, Pauler D K, Goodman P J, et al. Prevalence of prostate cancer among men with a prostate-specific antigen level < or =4.0 ng per milliliter. [see comment][erratum appears in N Engl J. Med. 2004 Sep. 30; 351(14):1470], New England Journal of Medicine 2004; 350: 2239-46.
4. Djavan B, Remzi M, Schulman C C, Marberger M, Zlotta A R. Repeat prostate biopsy: who, how and when? A review. Eur Urol 2002; 42: 93-103.
5. Epstein J I, Herawi M. Prostate needle biopsies containing prostatic intraepithelial neoplasia or atypical foci suspicious for carcinoma: implications for patient care. J Urol 2006; 175: 820-34.
6. Egevad L, Granfors T, Karlberg L, Bergh A, Stattin P. Prognostic value of the Gleason score in prostate cancer. BJU International 2002; 89: 538-42.
7. Pinthus J H, Witkos M, Fleshner N E, et al. Prostate cancers scored as Gleason 6 on prostate biopsy are frequently Gleason 7 tumors at radical prostatectomy: implication on outcome. Journal of Urology 2006; 176: 979-84.
8. D'Amico A V, Whittington R, Malkowicz S B, Fondurulia J, Chen M H, Kaplan I, Beard C J, Tomaszewski J E, Renshaw A A, Wein A, Coleman C N. Pretreatment nomogram for prostate-specific antigen recurrence after radical prostatectomy or external-beam radiation therapy for clinically localized prostate cancer. J Clin Oncol. 1999 January; 17(1):168-72.
9. Parikh K, Peppelenbosch M P: Kinome profiling of clinical cancer specimens. Cancer Res, 70(7):2575-2578.
10. Henson D E: Back to the drawing board on immunohistochemistry and predictive factors. J Natl Cancer Inst 2005, 97(24):1796-1797.
11. Rudiger T, Hofler H, Kreipe H H, Nizze H, Pfeifer U, Stein H, Dallenbach F E, Fischer H P, Mengel M, von Wasielewski R et al: Quality assurance in immunohistochemistry: results of an interlaboratory trial involving 172 pathologists. Am J Surg Pathol 2002, 26(7):873-882.
12. Rhodes A, Jasani B, Balaton A J, Miller K D: Immunohistochemical demonstration of estrogen and progesterone receptors: correlation of standards achieved on in house tumours with that achieved on external quality assessment material in over 150 laboratories from 26 countries. J Clin Pathol 2000, 53(4):292-301.
13. Rhodes A, Borthwick D, Sykes R, Al-Sam S, Paradiso A: The use of cell line standards to reduce HER-2/neu assay variation in multiple European cancer centers and the potential of automated image analysis to provide for more accurate cut points for predicting clinical response to trastuzumab. Am J Clin Pathol 2004, 122(1):51-60.
14. Fox C H, Johnson F B, Whiting J. Roller P P: Formaldehyde fixation. J Histochem Cytochem 1985, 33(8):845-853.
15. Dimenstein I B: A Pragmatic Approach to Formalin Safety in Anatomical Pathology. Labmed 2009, 40:740-746.
16. Gillespie J W, Best C J, Bichsel V E, Cole K A, Greenhut S F, Hewitt S M, Ahram M, Gathright Y B, Merino M J, Strausberg R L et al: Evaluation of non-formalin tissue fixation for molecular profiling studies. Am J Pathol 2002, 160(2):449-457.
17. Ahram M, Flaig M J. Gillespie J W, Duray P H, Linehan W M, Ornstein D K, Niu S, Zhao Y, Petricoin E F, 3rd, Emmert-Buck M R: Evaluation of ethanol-fixed, paraffin-embedded tissues for proteomic applications. Proteomics 2003, 3(4):413-421.
18. Cox M L, Schray C L, Luster C N, Stewart Z S, Korytko P J, K N M K, Paulauskis J D, Dunstan R W: Assessment of fixatives, fixation, and tissue processing on morphology and RNA integrity. Exp Mol Pathol 2006, 80(2):183-191.
19. Jemal A, Siegel R, Xu J, Ward E: Cancer statistics, 2010. CA Cancer J Clin, 60(5):277-300.
20. Hernandez J, Thompson I M: Prostate-specific antigen: a review of the validation of the most commonly used cancer biomarker. Cancer 2004, 101(5):894-904.
21. Schroder F H, Hugosson J, Roobol M J, Tammela T L, Ciatto S, Nelen V, Kwiatkowski M, Lujan M, Lilja H. Zappa M et al: Screening and prostate-cancer mortality in a randomized European study. N Engl J Med 2009, 360(13):1320-1328.
22. Andriole G L, Crawford E D, Grubb R L, 3rd, Buys S S, Chia D, Church T R, Fouad M N, Gelmann E P, Kvale P A, Reding D J et al: Mortality results from a randomized prostate-cancer screening trial. N Engl J Med 2009, 360(13):1310-1319.
23. Bastian P J, Carter B H, Bjartell A, Seitz M, Stanislaus P, Montorsi F, Stief C G, Schroder F: Insignificant prostate cancer and active surveillance: from definition to clinical implications. Eur Urol 2009, 55(6):1321-1330.
24. Miocinovic R. Jones J S, Pujara A C, Klein E A, Stephenson A J: Acceptance and Durability of Surveillance as a Management Choice in Men with Screen Detected, Low Risk Prostate Cancer: Improved Outcomes with Stringent Enrollment Criteria. Urology 2011.
25. O'Toole S A, Selinger C I, Millar E K, Lum T, Beith J M: Molecular assays in breast cancer pathology. Pathology, 43(2):116-127.
26. Kelley R K, Van Bebber S L, Phillips K A, Venook A P: Personalized medicine and oncology practice guidelines: a case study of contemporary biomarkers in colorectal cancer. J Natl Compr Cane Netw, 9(1):13-25.
27. Turaga K, Acs G, Laronga C: Gene expression profiling in breast cancer. Cancer Control, 17(3):177-182.
28. Troyer D A, Lucia M S, de Bruine A P, Mendez Meza R, Baldewijns M M, Dunscomb, Van Engeland M, McAskill T, Bierau K, Louwagie J et al: Prostate cancer detected by methylated gene markers in histopathologically cancer negative tissues from men with subsequent positive biopsies. Cancer Epidemiol Biomarkers Prev 2009, 18(10):2717-2722.
29. Baden J, Green G, Painter J, Curtin K, Markiewicz J, Jones J, Astacio T, Canning S, Quijano J, Guinto W et al: Multicenter evaluation of an investigational prostate cancer methylation assay. J Urol 2009, 182(3):1186-1193.
30. Hoque M O: DNA methylation changes in prostate cancer: current developments and future clinical implementation. Expert Rev Mol Diagn 2009, 9(3):243-257.
31. Mucci L A, Pawitan Y, Demichelis F, Fall K, Stark J R, Adami H O, Andersson S O, Andren O, Eisenstein A, Holmberg L et al: Testing a multigene signature of prostate cancer death in the Swedish Watchful Waiting Cohort. Cancer Epidemiol Biomarkers Prev 2008, 17(7):1682-1688.
32. Donovan M J, Khan F M, Fernandez G, Mesa Tejada R, Sapir M, Zubek V B, Powell D, Fogarasi S, Vengrenyuk Y, Teverovskiy M et al: Personalized prediction of tumor response and cancer progression on prostate needle biopsy. J Urol 2009, 182(1):125-132.
33. Donovan M J, Hamann S, Clayton M, Khan F M, Sapir M, Bayer Zubek V, Fernandez G, Mesa Tejada R, Teverovskiy M. Reuter V E et al: Systems pathology approach for the prediction of prostate cancer progression after radical prostatectomy. J Clin Oncol 2008, 26(24):3923-3929.
34. Sreekumar A, Poisson L M, Rajendiran T M, Khan A P, Cao Q, Yu J, Laxman B, Mehra R, Lonigro R J, Li Y et al: Metabolomic profiles delineate potential role for sarcosine in prostate cancer progression. Nature 2009, 457(7231):910 914.
35. Tessem M B, Swanson M G, Keshari K R, Albers M J, Joun D, Tabatabai Z L, Simko J P, Shinohara K, Nelson S J, Vigneron D B et al: Evaluation of lactate and alanine as metabolic biomarkers of prostate cancer using 1H HR-MAS spectroscopy of biopsy tissues. Magn Reson Med 2008, 60(3):510-516.
36. Swanson M G, Keshari K R, Tabatabai Z L, Simko J P, Shinohara K, Carroll P R, Zektzer A S, Kurhanewicz J: Quantification of choline- and ethanolamine-containing metabolites in human prostate tissues using 1H HR-MAS total correlation spectroscopy. Magn Reson Med 2008, 60(1):33-40.
37. Serkova N J, Gamito E J, Jones R H, O'Donnell C, Brown J L, Green S, Sullivan H, Hedlund T, Crawford E D: The metabolites citrate, myo-inositol, and spermine are potential age-independent markers of prostate cancer in human expressed prostatic secretions. Prostate 2008, 68(6):620-628.
38. Mueller-Lisse U G, Scherr M K: Proton MR spectroscopy of the prostate. Eur J Radiol 2007, 63(3):351-360.
39. Jordan K W, Cheng L L: NMR-based metabolomics approach to target biomarkers for human prostate cancer. Expert Rev Proteomics 2007, 4(3):389-400.
40. Costello L C, Franklin R B: The clinical relevance of the metabolism of prostate cancer; zinc and tumor suppression: connecting the dots. Mol Cancer 2006, 5:17.
41. Dotan E, Cohen S J, Alpaugh K R, Meropol N J: Circulating tumor cells: evolving evidence and future challenges. Oncologist 2009, 14(11): 1070-1082.
42. Eschwege P, Moutereau S, Droupy S, Douard R, Gala J L, Benoit G, Conti M, Manivet P, Loric S: Prognostic value of prostate circulating cells detection in prostate cancer patients: a prospective study. Br J Cancer 2009, 100(4):608-610.
43. Okegawa T, Nutahara K, Higashihara E: Prognostic significance of circulating tumor cells in patients with hormone refractory prostate cancer. J Urol 2009, 181(3):1091-1097.
44. Davis J W, Nakanishi H, Kumar V S, Bhadkamkar V A, McCormack R, Fritsche H A, Handy B, Gornet T, Babaian R J: Circulating tumor cells in peripheral blood samples from patients with increased serum prostate specific antigen: initial results in early prostate cancer. J Urol 2008, 179(6):2187-2191; discussion 2191.
45. Danila D C, Heller G, Gignac G A, Gonzalez-Espinoza R, Anand A, Tanaka E, Lilja H, Schwartz L, Larson S, Fleisher M et al: Circulating tumor cell number and prognosis in progressive castration-resistant prostate cancer. Clin Cancer Res 2007, 13(23):7053-7058.
46. Evans A M, DeHaven C D, Barrett T, Mitchell M, Milgram E: Integrated, nontargeted ultrahigh performance liquid chromatography/electrospray ionization tandem mass spectrometry platform for the identification and relative quantification of the small-molecule complement of biological systems. Anal Chem 2009, 81(16):6656-6667.
47. Dehaven C D, Evans A M, Dai H, Lawton K A: Organization of GCiMS and LC/MS metabolomics data into chemical libraries. J Cheminform 2010, 2(1):9.
48. Crehange G, Parfait S, Liegard M, Maingon P, Ben Salem D, Cochet A, Funes de la Vega M, Cormier L, Bonnetain F, Mirjolet C et al: Tumor Volume and Metabolism of Prostate Cancer Determined by Proton Magnetic Resonance Spectroscopic Imaging at 3T Without Endorectal Coil Reveal Potential Clinical Implications in the Context of Radiation Oncology. Int J Radiat Oncol Biol Phys 2010.
49. Zakian K L, Shukla Dave A, Ackerstaff E, Hricak H, Koutcher J A: 1H magnetic resonance spectroscopy of prostate cancer: biomarkers for tumor characterization. Cancer Biomark 2008, 4(45):263-276.
50. Swanson M G, Zektzer A S, Tabatabai Z L, Simko J, Jarso S, Keshari K R, Schmitt L, Carroll P R, Shinohara K, Vigneron D B et al: Quantitative analysis of prostate metabolites using 1H HR MAS spectroscopy. Magn Reson Med 2006, 55(6):1257-1264.
51. Schipper R G, Romijn J C, Cuijpers V M, Verhofstad A A: Polyamines and prostatic cancer. Biochem Soc Trans 2003, 31(2):375-380.
52. Oyama T, Allsbrook W C, Jr., Kurokawa K, Matsuda H, Segawa A, Sano T, Suzuki K, Epstein J I: A comparison of interobserver reproducibility of Gleason grading of prostatic carcinoma in Japan and the United States. Arch Pathol Lab Med 2005, 129(8):1004-1010.
53. Netto G J, Eisenberger M, Epstein J I: Interobserver Variability in Histologic Evaluation of Radical Prostatectomy Between Central and Local Pathologists: Findings of TAX 3501 Multinational Clinical Trial. Urology 2010.
54. Allsbrook W C, Jr., Mangold K A, Johnson M H, Lane R B, Lane C G, Epstein J I: Interobserver reproducibility of Gleason grading of prostatic carcinoma: general pathologist. Hum Pathol 2001, 32(1):81-88.
55. Allsbrook W C, Jr., Mangold K A, Johnson M H, Lane R B, Lane C G, Amin M B, Bostwick D G, Humphrey P A, Jones E C, Reuter V E et al: Interobserver reproducibility of Gleason grading of prostatic carcinoma: urologic pathologists. Hum Pathol 2001, 32(1):74-80.
56. Paik S, Shak S, Tang G, Kim C, Baker J, Cronin M, Baehner F L, Walker M G, Watson D, Park T et al: A multigene assay to predict recurrence of tamoxifen treated, node negative breast cancer. N Engl J Med 2004, 351(27):2817-2826.
57. Hewitt S M, Lewis F A, Cao Y, Conrad R C, Cronin M, Danenberg K D, Goralski T J, Langmore J P, Raja R G, Williams P M et al: Tissue handling and specimen preparation in surgical pathology: issues concerning the recovery of nucleic acids from formalin fixed, paraffin embedded tissue. Arch Pathol Lab Med 2008, 132(12):1929-1935.
58. Tuck M K, Chan D W, Chia D, Godwin A K, Grizzle W E, Krueger K E, Rom W, Sanda M, Sorbara L, Stass S et al: Standard operating procedures for serum and plasma collection: early detection research network consensus statement standard operating procedure integration working group. J Proteome Res 2009, 8(1):113-117.
59. McLerran D, Grizzle W E, Feng Z, Thompson I M, Bigbee W L, Cazares L H, Chan D W, Dahlgren J, Diaz J, Kagan J et al: SELDI TOF MS whole serum proteomic profiling with IMAC surface does not reliably detect prostate cancer. Clin Chem 2008, 54(1):53-60.
60. Vandromme M J, Umphrey H, Krontiras H: Image guided methods for biopsy of suspicious breast lesions. J Surg Oncol. 103(4):299-305.
61. Solomon S B, Zakowski M F, Pao W, Thornton R H, Ladanyi M, Kris M G, Rusch V W, Rizvi N A: Core needle lung biopsy specimens: adequacy for EGFR and KRAS mutational analysis. AJR Am J Roentgenol, 194(1):266-269.
62. Piccart Gebhart M J, Procter M, Leyland Jones B, Goldhirsch A, Untch M, Smith I, Gianni L, Baselga J, Bell R, Jackisch C et al: Trastuzumab after adjuvant chemotherapy in HER2 positive breast cancer. N Engl J Med 2005, 353(16):1659-1672.
63. Diamond J, Anderson N, Bartels P, Montironi R, and Hamilton P. (2004) The use of morphological characteristics and texture analysis in the identification of tissue composition in prostatic neoplasia, Human Pathology 35: 1121-1131
64. Tabesh A, Teverovskiy M, Pang H-Y, Kumar V P, Verbel D, Kotsianti A, Saidi O, (2007) Multifeature prostate cancer diagnosis and Gleason grading of histological images, IEEE Trans. Med. Imag. 26: 1366-1378
65. Huang P-W, Lee C-H. (2009) Automatic Classification for Pathological Prostate Images Based on Fractal Analysis,” IEEE Trans on Medical Imaging 28: 1037-1050
66. Shuster et al. “Molecular preservation by extraction and fixation, mPREF: a method for small molecule biomarker analysis and histology on exactly the same tissue.” BMC Clinical Pathology 2011, 11:14
67. Sandblom G, Dufmats M, Varenhorst E. (2000) Longterm survival in a Swedish population based cohort of men with prostate cancer. Urology 56: 442-447
68. Kramer B S, Croswell J M. Cancer screening: the clash of science and intuition. (2009) Annu Rev Med 60:125-137
69. Welch H G, Black W C. (2010) Overdiagnosis in cancer. J Natl Cancer Inst. 102:605-613.
70. Eckersberger E, Finkelstein J, Sadri H, Margreiter M, Taneja S S, Lepor H, Djavan B. (2009) Screening for Prostate Cancer: A review of the ERSPC and PLCO Trials. Rev Urol 2009 11:127-133
71. Berger M F, et al. (2011) The genomic complexity of primary human prostate cancer. Nature 470:214-220

1. A method for screening for prostate cancer in a subject comprising the steps of:
(a) providing a biological sample from a subject;
(b) detecting at least one biomarker in said sample, said biomarker selected from the group consisting of betaine, malate, proline, N-acetylaspartate, uracil, xanthine, cysteine, alanine, and N-acetylglucosamine; and
(c) correlating said detection with a status of prostate cancer or no prostate cancer.

2. A method for screening for prostate cancer in a subject comprising the steps of:
(a) providing a biological sample from a subject;
(b) detecting at least one biomarker in said sample, said biomarker selected from the group consisting of betaine, malate, proline, uracil, xanthine, cysteine, alanine, and N-acetylglucosamine; and
(c) correlating said detection with a status of prostate cancer or no prostate cancer.

3. The method of claim 1 wherein said detecting at least one biomarker is performed by mass spectrometry.

4. The method of claim 1, wherein the biological sample is selected from the group consisting of biological fluid and tissue.

5. The method according to claim 4, wherein the biological fluid is whole blood, serum, plasma, or urine.

6. The method according to claim 4, wherein the tissue is a prostate tissue sample.

7. The method of claim 1, wherein the biological sample is contacted with a solvent capable of extracting the at least one biomarker.

8. The method according to claim 7, wherein the solvent is methanol or ethanol.

9. A method of diagnosing prostate cancer in a subject, comprising the steps of:
(a) obtaining one or more test samples from a subject;
(b) detecting at least one biomarker in the one or more test samples, wherein the biomarker is selected from: betaine, malate, proline, N-acetylaspartate, uracil, xanthine, cysteine, alanine, and N-acetylglucosamine;
(c) quantitating the amount of the at least one biomarker; and
(d) correlating the quantitation of the at least one biomarker with a diagnosis of prostate cancer.

10. A method of diagnosing prostate cancer in a subject, comprising the steps of:
(a) obtaining one or more test samples from a subject;
(b) detecting at least one biomarker in the one or more test samples, wherein the biomarker is selected from: betaine, malate, proline, uracil, xanthine, cysteine, alanine, and N-acetylglucosamine;
(c) quantitating the amount of the at least one biomarker; and
(d) correlating the quantitation of the at least one biomarker with a diagnosis of prostate cancer, wherein the correlation takes into account the amount of the at least one biomarker in the one or more test samples compared to a control amount of the at least one biomarker.

11. The method of claim 10 wherein the correlation takes into account the amount of the at least one biomarker in the one or more test samples compared to a control amount of the at least one biomarker.

12. The method of claim 10 wherein the test sample is selected from the group consisting of urine, whole blood, serum, plasma, and prostate tissue.

13. A method of monitoring the effect of a prostate cancer drug or therapy on a subject comprising:
(a) providing a biological sample from the subject;
(b) contacting the biological sample with a solvent capable of extracting at least one prostate cancer biomarker selected from the group consisting of betaine, malate, proline, N-acetylaspartate, uracil, xanthine, cysteine, alanine, and N-acetylglucosamine;
(c) quantitating the amount of the at least one prostate cancer biomarker;
(d) providing the subject with an anti-prostate cancer drug or therapy;
(e) quantitating the amount of the at least one prostate cancer biomarker using steps (a) and (b); and
(f) correlating the two measurements with a diagnosis that the prostate cancer is regressing or progressing.

14. A multiplexed assay for screening for prostate cancer in a subject comprising the steps of:
(a) providing a biological sample from a subject;
(b) quantitating at least two or more biomarkers in said sample, said biomarkers selected from the group consisting of betaine, malate, proline, N-acetylaspartate, uracil, xanthine, cysteine, alanine, and N-acetylglucosamine;
(c) correlating said quantitation with a status of prostate cancer or no prostate cancer.

15. The method of claim 14 wherein said quantitating at least two or more biomarkers is performed by liquid chromatography in tandem with mass spectrometry.

16. The method of claim 14, wherein the biological sample is selected from the group consisting of biological fluid and tissue.

17. The method according to claim 16, wherein the biological fluid is whole blood, serum, plasma, or urine.

18. The method according to claim 16, wherein the tissue is a prostate tissue sample.

19. The method according to claim 14, wherein the biomarkers betaine, malate, proline, N-acetylaspartate, uracil, xanthine, cysteine, alanine, and N-acetylglucosamine are quantitated in the same assay.

20. The method according to claim 14, wherein the biomarkers betaine, malate, proline, uracil, xanthine, cysteine, alanine, and N-acetylglucosamine are quantitated in the same assay.

21. A multiplexed method for detecting prostate cancer in a subject comprising the steps of:
(a) providing a biological sample from the subject;
(b) contacting the biological sample with a solvent capable of extracting two or more prostate cancer biomarkers selected from the group consisting of betaine, malate, proline, N-acetylaspartate, uracil, xanthine, cysteine, alanine, and N-acetylglucosamine;
(c) quantitating the amount of the two or more biomarkers present in the biological sample; and
(d) correlating the amount of the two or more biomarkers with the presence or absence of prostate cancer.

22. The method of claim 21, wherein the quantitating differentiates between different stages of prostate cancer.

23. The method of claim 21, wherein the quantitating is part of a diagnosis or prognosis of prostate cancer in the subject.

24. The method according to claim 21, wherein the solvent is methanol or ethanol.

25. The method of claim 21, further comprising the step of performing additional histological analysis on the extracted biological sample.

26. A method of diagnosing prostate cancer in a subject, comprising the steps of:
(a) obtaining one or more test samples from a subject;
(b) detecting at least one biomarker in the one or more test samples, wherein the biomarker is selected from the group consisting of betaine, malate, proline, uracil, xanthine, cysteine, alanine, and N-acetylglucosamine;
(c) quantitating the amount of the at least one biomarker;
(d) determining the Gleason score of the one or more test samples;
(e) correlating the quantitation of the at least one biomarker and the Gleason score with a relative risk of T2 versus T3 prostate cancer.

27. A kit for diagnosing prostate cancer in a subject comprising:
(a) a vial for collecting a biological sample from the subject;
(b) a solvent for extracting biomarkers from the biological sample, the biomarkers selected from the group consisting of betaine, malate, proline, N-acetylaspartate, uracil, xanthine, cysteine, alanine, and N-acetylglucosamine;
(c) instructions for performing the extraction of the biomarkers;
(d) instructions for quantitating one or more of the biomarkers;
(f) instructions for correlating the quantitation of the one or more biomarkers to a diagnosis of prostate cancer or normal.
